SEQUENCE LISTING (4 sequences)

SEQ ID NO: 1 - DNA, 21 bp, Artificial Sequence
  Description of Artificial Sequence  Synthetic Primer
ggcaccgacgactacatctac

SEQ ID NO: 2 - DNA, 20 bp, Artificial Sequence
  Description of Artificial Sequence  Synthetic Primer
caattttgcacgtccatccc

SEQ ID NO: 3 - DNA, 20 bp, Artificial Sequence
  Description of Artificial Sequence  Synthetic Primer
cggccaggtcatcactattg

SEQ ID NO: 4 - DNA, 20 bp, Artificial Sequence
  Description of Artificial Sequence  Synthetic Primer
actcctgcttgctgatccac

(19) United States
(12) Patent Application Publication
(10) Pub. No.: US 2004/0014721 A1
(43) Pub. Date: Jan. 22, 2004

(54) Method for using tethered bis(polyhydroxyphenyls) and O-alkyl derivatives thereof in treating inflammatory conditions of the central nervous system

(75) Inventors: Kenneth Hensley, Oklahoma City, OK (US); Robert A. Floyd, Oklahoma City, OK (US)

Correspondence Address: FULBRIGHT & JAWORSKI L.L.P., 600 CONGRESS AVE., SUITE 2400, AUSTIN, TX, 78701, US

(73) Assignee: Oklahoma Medical Research Foundation

(21) Appl. No.: 10/455,235

(22) Filed: Jun. 5, 2003

Related U.S. Application Data
(60) Provisional application No. 60/387,374, filed on Jun. 10, 2002.

(51) Int. Cl. A61K 31/69; A61K 31/075

(52) U.S. Cl. 514/64; 514/721

(57) ABSTRACT

The present invention involves the use of tethered bis(polyhydroxyphenyl) compounds to slow the progression of neurological diseases in which pro-inflammatory cytokine stimulation of microglial cells is reasonably anticipated to make a significant contribution to disease pathology. Diseases for which this is the case include amyotrophic lateral sclerosis (ALS) and other motor neuron diseases (MNDs) of similar clinical presentation; Parkinson's disease (PD); Alzheimer's disease (AD); spino-bulbar atrophy; (SBA); Huntington's disease (HD); myasthenia gravis (MG); multiple sclerosis (MS); HIV-associated dementia; fronto-temporal dementia (FTD); stroke; encephalomyelitis; traumatic brain injury; age-related retinal degeneration; and other neurological diseases possessing microglial activation as a contributing pathological feature. Specific examples are presented where the tethered bis(polyhydroxyphenyl) compound is resveratrol; piceatannol; nordihydroguaiaretic acid (NDGA); curcumin, or sesamin.

The present application claims priority to co-pending U.S. Provisional Patent Application Serial No. 60/387,374 filed on Jun. 10, 2002. The entire text of the above-referenced disclosure is specifically incorporated herein by reference without disclaimer.

The government owns rights in the present invention pursuant to grant number RO3-AG20783 from the National Institutes of Aging.

### BACKGROUND OF THE INVENTION

1. Field of the Invention

The present invention relates generally to the fields of pharmacology and immunological pharmacotherapy. More particularly, it concerns methods for treating neurological diseases, including but not limited to neurological diseases exhibiting microglial activation as a contributing pathological feature. The present invention also concerns methods of treating other diseases such as inflammatory diseases and benign or malignant hyperplasias that involve components of pro-inflammatory cytokine action on macrophage-like cells.

2. Description of Related Art

Most or all neurological diseases share a common pathological feature: the activation of microglia, which are specialized myeloid (macrophage-like) cells in the central nervous system (CNS). For example, HLA-DR reactive microglia proliferate in regions of the Alzheimer's-diseased (AD) brain most dramatically affected by histopathological hallmarks of the disease (Wisniewski et al., 1990; Hensley et al., 1995). Similar microglial proliferation is observed in the spinal cord of patients with ALS (amyotrophic lateral sclerosis) (Hall et al., 1998; Alexianu et al., 2001); in the diseased Parkinsonian brain (Vila et al., 2001); in the brains of patients with HIV (Kaul et al., 2001); and in post-traumatic or post-ischemic brain tissue (Floyd et al., 2000). Thus, microglial responsitivity is common to most, if not all, neurodegenerative conditions. Some microglial functions are beneficial, for instance, in the clearing of apoptotic cells and the resolution of injury. Exacerbated or chronic microglial activation, on the other hand, can damage neurons through direct and indirect action involving overproduction of reactive oxygen and reactive nitrogen species (ROS and RNS), and the propagation of inflammatory cytokine cascades. When activated, microglia synthesize potential neurotoxins such as reactive oxygen species (ROS, including but not restricted to oxygen-centered free radicals); reactive nitrogen species (RNS, including but not restricted to nitric oxide and derived nitrogen oxides); and pro-inflammatory cytokines (including but not restricted to interleukin 1 (IL1α and IL1β), interferon gamma (IFNγ) and tumor necrosis factor alpha (TNFα) (Colton et al., 1994; Meda et al., 1995). In most cases, it is not clear exactly why microglia become activated, and few strategies have been proposed and tested that offer a clear means by which to suppress the conversion of microglia from an innocuous quiescent phenotype to an active and potentially neurotoxic phenotype. The development of such means would require discovery or invention and validation of small molecules that could inhibit microglial activation caused by multiple stimuli including exposure to pro-inflammatory cytokines (especially IL1β and TNFα) as well as immunoglobulins (especially IgG and autoantigen complexes). Such molecules would have to be permeable across the blood brain barrier to a degree that would allow CNS accumulation of the active compounds in sufficient concentration for bioactivity; and they would have to be essentially nontoxic to neurons and peripheral tissues.

Currently, there are very few treatment options for neuroinflammatory diseases including Alzheimer's disease (AD), Huntington's disease (HD), Parkinson's disease, and amyotrophic lateral sclerosis (ALS). The few current existing therapeutic modalities treat only the symptoms rather than the root causes of the disease. Several currently prescribed drugs specifically developed for AD are either cholinesterase inhibitors, or cytoskeleton-acting agents (Knopman, 2001). The only standard therapy for PD is dopamine replacement/augmentation (Damnis, 2002). The only drug currently approved for ALS, riluzole, is an anti-excitotoxicant that antagonizes NMDA receptors (Miller, 2001). There are no currently approved drugs for treating HD, although neuroleptics are sometimes used for symptoms (McMurray, 2001). Thus, there is a need for novel and non-obvious microglial suppressors for the treatment of neurological diseases, including inflammatory diseases and cancers or hyperplasias.

### SUMMARY OF THE INVENTION

The present invention provides bis(polyhydroxyphenyl) compounds (also known as dicathechols) and O-alkyl derivatives thereof for treating neurological diseases and other diseases such as inflammatory diseases, and cancers or hyperplasias that involve some components of pro-inflammatory cytokine action on microglial or macrophage-like cells. Particularly, the present invention provides tethered bis(polyhydroxyphenyl) compounds which are superior to other, merely linked bis(polyhydroxyphenyls) with respect to their specific ability to suppress the biochemical effects of pro-inflammatory cytokines.

Thus, in the present invention, there is provided a method of inhibiting an inflammatory disease in a subject comprising providing to the subject an effective amount of tethered bis(polyhydroxyphenyl) compounds, or O-alkyl derivatives thereof. The tethered bis(polyhydroxyphenyl) compounds of the present invention have the general formula:

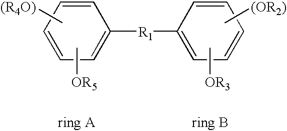

In particular embodiments, R₁ is an alkyl chain of at least 2 and less that 10 carbon units in length, and R₂-R₅ are —H atoms or alkyl chains comprising one or more carbon atoms. R₁ may comprise structural motifs selected from C═C bonds; alkynes; amide ester, ether or sulfide linkages; intervening ring structures; ketone moieties; or halogenated side chain. In another particular embodiment, R₂-R₅ may further comprise of the group selected from halogens, carbonyl groups, boronate esters and closed ring structures. In yet another particular embodiment, at least one of OR₄ and OR₅ and at least one of OR₂ and OR₃ is a hydroxyl or alkoxyl group. In further embodiments, the tethered R₁ is a branched chain hydrocarbon and at least three of OR₂-OR₅ are hydroxyl or alkoxyl groups.

In particular embodiments of the present invention, the tethered bis(polyhydroxyphenyl) compound is nordihydroguaiaretic acid (NDGA) or O-alkyl derivatives thereof or pro-drugs of the same; piceatannol or O-alkyl derivatives thereof or pro-drugs of the same; resveratrol or O-alkyl derivatives thereof or pro-drugs of the same; rooperol or O-alkyl derivatives thereof or pro-drugs of the same; rosmarinic acid or O-alkyl derivatives thereof or pro-drugs of the same; a tyrphostin comprising two phenolic ring structures, or O-alkyl derivatives thereof or pro-drugs of the same; butein or O-alkyl derivatives thereof or pro-drugs of the same; or curcumin, or reduced curcumin such as dihydrocurcumin or tetrahydrocurcumin, or O-alkyl derivatives thereof or pro-drugs of the same; or sesamin, or sesame compositions such as sesame oil or sesame seed extracts, or O-alkyl derivatives thereof or pro-drugs of the same.

In still yet another embodiment, the bis(polyhydroxyphenyl) compounds or O-alkyl derivatives thereof are used to treat neurological diseases, such as those involving pro-inflammatory cytokine stimulation of a microglial cell, a neuron, a macrophage type cell, a Kupffer cell, Mueller cell or other myeloid cell. In further embodiments of the present invention, the neurological disease is: amyotrophic lateral sclerosis (ALS) (familial or sporadic); motor neuron disease (MND) of similar clinical presentation to ALS; Alzheimer's disease (AD); Parkinson's disease (PD); multiple sclerosis (MS); myasthenia gravis (MG); Huntington's disease (HD); spinal-bulbar atrophy (SBA); frontal-temporal dementia (FTD); stroke (ischemia-reperfusion injury of the brain); traumatic brain injury, encephalomyelitis or meningitis; HIV-associated dementia or HIV-associated inflammatory diseases; or age-related retinal degeneration.

The present invention also embodies a method of treating inflammatory diseases or hyperplasias in a subject comprising providing to the subject an effective amount of a bis(polyhydroxyphenyl) compound or O-alkyl derivatives thereof to inhibit pro-inflammatory cytokine action on macrophage-like cells. In other embodiments of the invention, the inflammatory disease is cancer or hyperplasia of the eyes, respiratory system, musculo-skeletal system, lymphatic system, reticulo-endothelial system, hepatic system, prostrate, breast, colon, reproductive, urinary or alimentary tract. In another embodiment, the inflammatory disease is chronic inflammatory or rheumatic diseases such as: arthritis, inflammatory or rheumatic diseases of the eye, or diseases of the respiratory or musculo-skeletal system, or alimentary tract.

In further embodiments, the present invention provides a method of treating a subject with neurological diseases, or hyperplasias comprising administering to the subject an effective amount of a bis(polyhydroxyphenyl) or O-alkyl derivatives thereof to inhibit microglial activation.

Administration of compounds of the present invention may be administered orally, subcutaneously, intrathecally, by inhalation, injection, microprojectile bombardment, intravenously, or topically.

The present invention further embodies a method for enhancing the efficacy of non-bis(polyhydroxyphenyl) neuropharmaceuticals comprising providing to a subject a non-bis(polyhydroxyphenyl) neuropharmaceutical, such as riluzole or minocycline, and a bis(polyhydroxyphenyl) or O-alkyl derivative thereof. In further embodiments, the non-bis(polyhydroxyphenyl) or bis(polyhydroxyphenyl) or O-alkyl derivative thereof is provided gradually over a time period of: greater than one minute; greater than ten minutes; greater than thirty minutes; greater than sixty minutes; greater than one-hundred twenty minutes; greater than four hours; greater than eight hours; greater than twelve eight hours; greater than twenty-four hours. In still further embodiments, the non-bis(polyhydroxyphenyl), or the bis(polyhydroxyphenyl) or O-alkyl derivative thereof, is provided more than once. In still yet another embodiment, the bis(polyhydroxyphenyl) or O-alkyl derivative thereof, is provided before, or at the same time, or after the non-bis(polyhydroxyphenyl). In other embodiments, the bis(polyhydroxyphenyl) or O-alkyl derivative thereof may be provided in combination with non-steroidal anti-inflammatory (NSAIDS) drugs.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B. TNF-α stimulates RNS production in EOC-20 microglial culture. FIG. 1A—TNF-α, LPS, IL1β, EGF at same concentration. FIG. 1B—TNF-α, LPS, IL1β, IL-6, IFNγ, IL6+IFNγ at varying concentrations.

FIG. 2. Potent inhibition of TNF-α stimulated microglial activation indicated by nitrite flux using NDGA, minocycline, and curcumin. Each point is the mean of 4 wells.

FIGS. 3A-3C. Unprocessed rotorod performance times of G93A-SOD-1 mice administered NDGA or vehicle beginning at 90 days of age (FIG. 3A). Net decline in rotorod motor functional ability of NDGA and vehicle-treated animals (FIG. 3B). FIG. 3C—Motor function decline in mice bearing human G93A-SOD1 transgenes (mean±SD;10/group).

FIG. 4. Linear regression analysis of motor functional decline in G93A-SOD1 mice treated with NGDA, or vehicle. Heavy lines represent best fit to data; light lines indicate 95% confidence intervals.

FIG. 5. Sesame oil improves performance of G93A-SOD1 mice afflicted with amyotrophic lateral sclerosis (ALS).

FIG. 6. Submicromolar concentrations of NGDA effectively inhibits prostaglandin E2 (PGE2) in TNFα-stimulated EOC-20 cells.

FIG. 7. Enhancement of TNFα-stimulated microglial nitrite production by LTB₄. Bars represent mean±S.D., n=4 wells each.

FIG. 8. G93A-SOD1 primary glial cultures are more sensitive to TNFα stimulation than are nontransgenic glial cultures. Symbols represent the mean±SD, N=4 wells at each point. *P<0.01 for the transgene effect by repeated measures ANOVA.

FIG. 9. An interpretive model of the proposed TNFα-5LOX signaling axis in microglia.

FIGS. 10A-10B. Improvement of prognosis in G93A-SOD1 mice by oral administration of NDGA beginning at 90 D. The drug extends median survival by 13 D. P<0.01 by logrank analysis; N=16 mice per group. FIG. 10A—rotarod times at 90 D. FIG. 10B—percent survival.

FIG. 11. Reduction of astrogliosis in G93A-SOD1 mice by oral NDGA. Lumbar spinal cord sections from nontransgenic (nonTg) or G93A-SOD1 mice were labeled with anti-5LOX antibody. NDGA significantly decreased the number of GFAP-positive astrocytes present in G93A-SOD1 lumbar sections from transgenic mice. The bar graph indicates mean±SD for cell counts (12 fields per section at 40× magnification; 0.23 mm per field). *P<0.001 by Mann-Whitney test.

FIG. 12. Semiquantitative RT-PCR analysis of 5LOX mRNA in spinal cords of 120 D old G93A-SOD1 and nontransgenic mice. The 5LOX gel image was obtained after 30 PCR cycles with ethidium bromide detection; actin was obtained at 24 cycles. Each lane represents one animal. The bar graph indicates mean±SD, N=7. *P=0.011 by Mann-Whitney test.

FIG. 13. 5LOX (the 80 kDa protein band) is co-immunoprecipitated with SOD1 in nontransgenic mouse spinal cord lysates. The left and middle lanes each represented three pooled mouse cords.

FIGS. 14A-14C. BIAcore data indicates binding of 5LOX to surface-immobilized SOD1. FIG. 14A—Idealized sensorgram output from a BIAcore experiment. FIG. 14B—Actual sensorgram showing an interaction between 5LOX (0.5 mg/mL) and immobilized SOD1. Note the very slow dissociation kinetics. FIG. 14C—5LOX binds SOD1 in a concentration-dependent fashion, whereas albumin displays negligible binding.

FIG. 15. SOD1 binds human 5LOX-coated microtiter plates, but not to BSA-coated surfaces. Each point represents mean±SD of 4 wells.

FIG. 16. Western blot analysis of 5LOX protein in cortical tissue from APP/PS1 mice and age-matched nontransgenic animals. Bars represent average values.

FIG. 17. Western blot analysis of 5LOX protein in cortical tissue from human AD-afflicted brain, and tissue from age- and postmortem matched nondemented subjects. Bars represent average values for the several data points. SMTG=superior and middle temporal gyrus from which tissue was extracted.

FIG. 18. NGDA protection against amyloid β-induced memory deficits (indicated by measurement of latency times) in a Morris water maze test.

FIGS. 19A-19B. Oral NDGA strongly protects against movement deficits caused by systemic injection of 3NP. Balance beam data was collected 12 H after the final 3NP injection. N=4-5 animals/group. P<0.05 for NDGA effects upon the rotarod task (FIG. 19A) by repeated measures ANOVA; P<0.05 for NDGA effects on balance test (FIG. 19B) by Student's t-test.

### DESCRIPTION OF ILLUSTRATIVE EMBODIMENTS

A limited number of studies have implicated specific tethered bis(polyhydroxyphenyls) as neuroprotective agents but have failed to consider the significance of microglial-driven neuroinflammation. To date, the only compound that has been well-tested in vivo under the operational assumption of microglial inhibitory action is minocycline, which is a tetracycline derivative not structurally related to linked or tethered bis(polyhydroxy)phenyls (Yijanheikki et al., 1999; Tikka et al., 2001; Chu et al., 2002; Chen et al., 2000; Walker et al., 1995).

The action of tethered bis(polyhydroxyphenyls), individually or as a group, with respect to direct inhibition of cytokine receptor tyrosine kinases (C-RTKs), or as a functionally distinct and superior class of microglial-suppressive agents, have not been previously considered. The inventors have performed detailed investigations aimed at defining the structural features of polyphenols that best predict efficacy in microglial inhibition. Consequently, they have identified a heretofore unappreciated structural grouping of organic molecules that are superior microglial inhibitors.

I. The Present Invention

The present invention provides a meaningful distinction between tethered bis(polyhydroxyphenyl) compounds (also called dicatechols) and those that are merely linked, but not tethered. The present invention further provides a functional relationship amongst compounds that were previously thought of as unrelated. For instance, a functional relationship between curcumin, NDGA, tyrphostin AG-575 and piceatannol has not been postulated since these compounds have been thought of as occupying functionally distinct chemical groupings. Importantly, the present invention excludes from the class of “tethered bis(polyhydroxyphenyls)” those molecules that are linked but not tethered, including flavonoids and isoflavonoids, which were previously considered in a similar context with the stilbene derivatives resveratrol and piceatannol (Chi et al., 2001). Particularly, the present invention provides tethered bis(polyhydroxyphenyls) which are superior to other, merely linked bis(polyhydroxyphenyls) with respect to their specific ability to suppress the biochemical effects of pro-inflammatory cytokines most notable TNFα; and further provides evidence to justify such assertion. The present invention specifically considers the optimum structural characteristics necessary to inhibit microglial activation in neurological disease, and show that tethered bis(polyhydroxyphenyl) compounds are superior in this respect to those that are merely linked. Also provided is evidence that tethered bis(polyhdroxyphenyl) compounds are generally superior to the benchmark microglial inactivator minocycline, which is a meaningful but non-obvious comparison whose results immediately imply a novel utility inherent to the class of tethered bis(polyhdyroxyphenyls).

Additionally, the present invention concerns methods for treating neurological diseases including but not limited to: amyotrophic lateral sclerosis (ALS) and other motor neuron diseases (MNDs) of similar clinical presentation; Parkinson's disease (PD); Alzheimer's disease (AD); spino-bulbar atrophy; (SBA); Huntington's disease (HD); myasthenia gravis (MG); multiple sclerosis (MS); HIV-associated dementia; fronto-temporal dementia (FTD); stroke; traumatic brain injury; age-related retinal degeneration; encephalomyelitis; and other neurological diseases possessing microglial activation as a contributing pathological feature. The present invention also provides a method of treating other diseases such as inflammatory diseases and cancers or hyperplasias that involve some components of pro-inflammatory cytokine action on macrophage-like cells.

II. Bis(polyhydroxyphenyls) Compounds

Several families of botanical natural products consist of two aromatic rings, both phenolic in nature, connected by a linkage group. Heretofore this broad classification of organic compounds is designated bis(polyhydroxyphenyls) or dicatechol to describe the relevant components of the chemical framework. Examples of bis(polyhydroxyphenyls) include flavones, flavanones, isoflavones and chalcones; specific tyrphostins containing two phenolic ring systems; hydroxylated stilbene derivatives such as resveratrol and piceatannol; and miscellaneous natural products including nordihydroguaiaretic acid (NDGA). When the two polyhydroxyphenyl groups are linked by a flexible carbonaceous chain (typically an alkyl chain of two or more atomic centers, containing or not containing other structural motifs such as C═C bonds, amide, sulfide, ester or ether linkages, or ketone moieties, the whole of which connects each ring exactly once), the structure can be accurately described as a tethered bis(polyhydroxyphenyl) or dicatechol. Thus, piceatannol, resveratrol and NDGA are tethered bis(polyhydroxyphenyls) and curcumin is an O-alkyl derivative of the class. Contrastingly, flavonoids and the like, which are linked by constraining ring systems, are not tethered.

TABLE 1
Bis(polyhydroxy) Phenyls

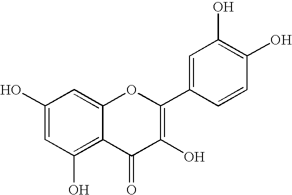

quercetin
(3′,4′,5,7-tetrahydroxy-flavone)

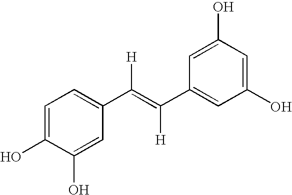

piceatannol
(3,3′,4′,5 -tetrahydroxy stilbene)

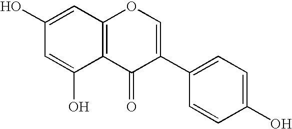

genistein
(4′,5,7-trihydroxy-isoflavone)

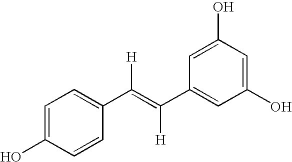

resveratrol
(3,4′,5-trihydroxy stilbene)

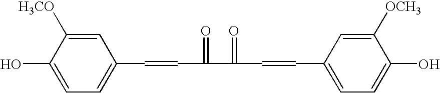

curcumin
(7-Bis (4-hydroxy-3-methoxyphenyl)-1,6-heptadiene-3,5-dione)

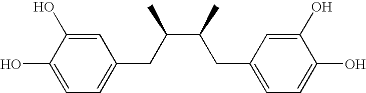

nordihydroguaiaretic
(acid [NDGA or 1,4-bis (3,4-Dihydroxyphenyl)-2,3-dimethylbutane])

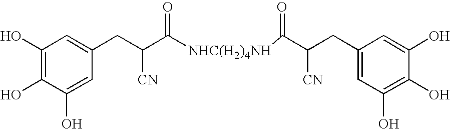

tyrphostin AG-575

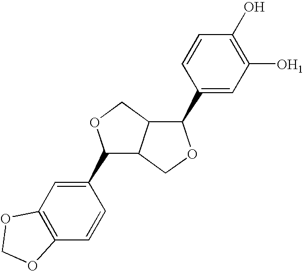

sesamin metabolite 1

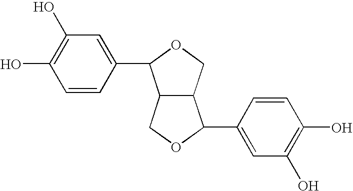

sesamin metabolite 2

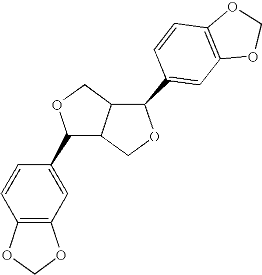

sesamin

Investigators have often failed entirely to draw distinctions between linked and tethered bis(polyhydroxyphenyls). For instance, Cho et al. (2000) studied extracts from the bark of Pinus maritima in a macrophage model. This is the source for piceatannol, as well as other flavonoids that are linked but not tethered. Cho et al. (2000) implicitly grouped all these compounds under the class “bioflavonoids”, without making a distinction between those that are tethered and those that are merely linked but not tethered. By failing to make a distinction between linked and tethered bis(polyhydroxyphenyls), previous investigators have failed to appreciate their superiority as anti-inflammatory agents of a functional subgrouping of natural products.

III. Functional Aspects of Bis(polyhydroxyphenyl) Compounds

Individual members of the class of bis(polyhydroxyphenyls) compounds defined by the inventors as “tethered bis(polyhydroxyphenyls)” have been investigated for a variety of bioactivities, usually involving anti-poliferative (anti-carcinogenic) effects but occasionally involving peripheral inflammatory situations. Rarely have individual members of the class of tethered bis(polyhydroxyphenyls) been investigated for neuroprotective action; and virtually no published work has evaluated these compounds in vivo in models that could be anticipated to involve microglial-driven events as a significant pathological component except that by Shishido et al. (2001) using linked bis(polyhydroxyphenyls). To date, the structural features that define the tethered bis(polyhydroxyphenyls) have not been enumerated and constrained in such a way to identify this classification of compounds as uniquely potent inhibitors of microglial activation (or of macrophage activation).

Specific members of the bis(polyhydroxyphenyl) class of compounds have been documented as having anticarcinogenic activity evidenced by their ability to slow cell proliferation (Birt et al., 2001; Miquel et al., 2002; Thakkar et al., 1993; Wolter et al., 2002; Wieder et al., 2001; Blum et al., 2000) and a smaller number reportedly have weak anti-inflammatory effects (Blum et al., 2000; Gazit et al., 1989; Park et al., 2000; Chi et al., 2001; Cho et al., 2000; Kageura et al., 2001). For the most part, previous attention has focused on the linked members (especially flavonoids) rather than the tethered members and these studies have largely focused on anti-carcinogenic effects (Birt et al., 2001). In most instances the mechanism of bioactivity is not known. Specific bis(polyhydroxyphenyls), such as specific tyrphostins, resveratrol and piceatannol are thought to inhibit growth factor receptor tyrosine kinases (GF-RTKs) associated with uncontrolled cellular proliferation (Thakkar et al., 1993; Wolter et al., 2002; Wieder et al., 2001; Blum et al., 2000; Gazit et al., 1989).

Much less work has focused on bis(polyhydroxyphenyl) effects on inflammatory diseases and virtually no work has focused on microglial biology per se. When bis(polyhydroxyphenyl) compounds have been studied in models of peripheral inflammation, attention has focused on the linked (but not tethered) subclass. Few studies have considered the ability of tethered bis(polyhydroxyphenyl) compounds to inhibit microglial-driven inflammatory reactions in neurodiseases. Resveratrol and NDGA have been studied with respect to their ability to inhibit peroxidase enzymes specifically cyclo-oxygenase (COX) and lipoxygenase (LOX) in macrophage cells, but are not currently documented to antagonize microglial signaling pathways initiated by pro-inflammatory cytokine binding to cytokine-receptor tyrosine kinases (C-RTKs).

1. Resveratrol

Trans-resveratrol has been found to play a role in protecting rodents against excitotoxic brain damage in vivo, after administration of the neurotoxin kainic acid (Virgili et al., 2000). However, similar treatment failed to protect neurons in vitro. Similarly, Gupta et al. (2002), demonstrated protective action of resveratrol against kainic acid-induced seizures and oxidative stress in rats. These effects were ascribed mostly to an antioxidant action and not to the anti-neuroinflammatory action of tethered bis(polyhydroxyphenyls). The ability of resveratrol to reduce infarct size in Long-Evans rats subjected to focal cerebral ischemia was also demonstrated by Huang et al. (2001). The results were ascribed to “anti-platelet aggregation activity, vasodilating effect, antioxidant property or by all mechanisms together” (Huang et al., 2001). The ability of resveratrol to inhibit peroxidase enzymes specifically cyclo-oxygenase (COX) and lipoxygenase (LPOX) in macrophage cells has also been demonstrated. Additionally, the ability of some stilbene derivatives from rhubarb to inhibit lipopolysaccharide-induced nitric oxide production in macrophages has been documented (Kageura et al., 2001).

2. Curcumin

Curcumin or herbal extracts containing curcumin have been proposed as inhibitors of inflammation or allergens (U.S. Pat. Nos. 6,235,287; 6,264,995) and of NFκB activation (U.S. Pat. No. 5,891,924). Curcumin antioxidants have been demonstrated to have benefits for cardiovascular disease and peripheral inflammation (i.e., psoriasis, liver injury; Miquel et al., 2002). The effect of curcumin on ethanol-induced brain damage in rats has also been found to be efficacious at reversing lipid peroxidation (Rajakrishnan et al., 1999). These beneficial effects were ascribed to “antioxidant and hypolipidaeimic action” but anti-neuro-inflammatory action was not explicitly considered nor were structural requirements for bioactivity defined. The ability of curcumin to reduce plaque-related pathology in a transgenic mouse model of Alzheimer's disease has also been demonstrated Lim et al. (2001). This study found that curcumin lowered protein oxidation and interleukin-1-beta, and suppressed microglial proliferation in neuronal layers but not adjacent to senile plaques. Similarly, the effect of curcumin in the reduction of age-associated damage caused by intracerebroventricular infusion of amyloid peptides has also been demonstrated (Frautschy et al., 2001). Hence, these studies considered neuroinflammatory features that are inhibited by curcumin, in addition to other putative mechanisms of action. However, both studies, focused on only one compound and therefore failed to identify the critical structural features of curcumin that define the activity of the molecule and that define the class of tethered bis(polyhydroxyphenyl) compounds.

3. Nordihydroguaiaretic Acid (NDGA)

NDGA has been studied as a neuroprotectant or an inhibitor of post-ischemic brain damage in an animal model of stroke, and found to be protective (Shishido et al., 2001). The presumptive mechanism of action of NDGA in this study was combined lipoxygenase activity and antioxidant effects; however, microglial suppressive effects were not explicitly considered (Kageura et al., 2001). The ability of NDGA to inhibit peroxidase enzymes specifically cyclo-oxygenase (COX) and lipoxygenase (LPOX) in macrophage cells, has also been demonstrated.

Nordihydroguaiaretic acid derivatives have also been proposed for treatment of HPV-induced cancer using in situ application (Huang et al., 2001). Lipoxygenase inhibitors have been proposed generically for use as anti-inflammatory or anti-allergy agents with possible utility in neurological disease (U.S. Pat. Nos. 4,708,964; 4,857,558; 5,047,593; 5,068,251; 5,208,262), and some bis(polyhydroxyphenyl) derivatives coincidentally do have lipoxygenase inhibiting action; however, it is noted by the present invention that lipoxygenase inhibiting activity is not sufficient to maximize activity of the bis(polyhydroxyphenyl) compounds.

4. Rooperol and Butein

Rooperol, is a specific tethered bis(polyhydroxyphenyl), with derivatives for use in treating specific inflammatory diseases of the bowel, colon, respiratory tract, skin and eyes (U.S. Pat. No. 5,569,649).

Butein is a another tethered bis(polyhydroxyphenyl) which has been shown to be a specific protein tyrosine kinase inhibitor. Butein has also been demonstrated to inhibited the epidermal growth factor (EGF)-stimulated auto-phosphotyrosine level of EGF receptor in some cells (Yang et al., 1998). This compound had also been shown to markedly suppress growth and induce cell death of cancer cells.

5. Sesamin

Sesamin is a major lignan in sesame oil, and its biological effects have been well documented. Sesamin has been shown to be a specific inhibitor of Δ5 desaturase (Shimizu et al., 1991), which catalyzes the conversion of dihomo-γ-linolenic acid to arachidonic acid, in both microorganisms and animals, and exerts hypocholesterolemic activity through the inhibition of cholesterol absorption and synthesis (Hirose et al., 1991). It has also been reported that sesamin prevents the damage to the liver caused by alcohol or carbon tetrachloride (Akimoto et al., 1993) and shows a suppressive effect against 7,12-dimethylbenz[α]anthracene-induced rat mammary carcinogenesis (Hirose et al., 1992) and antihypertensive effects (Matsumura et al., 1995; Kita et al., 1995; Matsumura et al., 1998; Nakano et al., 2002), although the mechanisms of action of this lignan remain unclear.

In sesame oil, lignans carrying a hydroxy group, that is, sesaminol, episesaminol, and sesamolinol, exhibit antioxidant activity (Osawa et al., 1985; Fukuda et al., 1985); however, sesamin as an antioxidant has not been evaluated clearly. The metabolized dicatechol products of sesamin in the liver after oral administration to rats were shown to be responsible for antioxidative properties observed Nakai et al. (2003). These antioxidative metabolites of sesamin have been isolated and structurally identified (see Table 1) but their anti-inflammatory action(s) has not been evaluated.

6. Other bis(polyhydroxyphenyls) Compounds

A number of bis(polyhydroxyphenyls) including both linked members (especially flavonoids) and the tethered member curcumin have been evaluated (Soliman et al., 1998). These compounds were studied with respect to their ability to inhibit nitric oxide production in C6 astrocyte culture exposed to lipopolysaccharide plus interferon gamma. However, the superiority of the tethered bis(polyhydroxyphenyl) compounds over other classes of natural compound were not discerned. Quercetin, morin and epicatechin gallate, which are linked but not tethered bis(polyhydroxyphenyls), were found to be superior to curcumin in the LPS-stimulated C6 astrocyte model system (Soliman et al., 1998). For example the IC₅₀ value for quercetin was 62 nM; for morin was 56 μM; and for epicatechin gallate was 10 μM; but the IC₅₀ value for curcumin was 72 μM ((Soliman et al., 1998); compare to the relative effects of quercetin vs. curcumin against TNFα-stimulated microglial activation in the present invention; Table II). Thus, the findings of Soliman et al. (1998) teach against the utility of the present invention. The failure of in this study to recognize the benefits of tethered bis(polyhydroxyphenyls) likely resulted from the nature of the stimulus: LPS is not a physiologically relevant stimulus in the central nervous system, except in special instances such as meningitis, so that compounds which inhibit C-RTK might likely fail to inhibit signaling pathways initiated by LPS. Likewise astrocytes (specifically, non-primary astrocyte cell lines, which may be carcinogenic and have altered signal processing pathways) are not the most relevant cell type to consider in the context of neuroinflammatory events. This study implicitly grouped tethered bis(polyhydroxyphenyl) compounds along with those that are merely linked but not tethered, under the rubric of “dietary-derived polyphenolic compounds”.

IV. Extraction and Purification Bis(polyhydroxyphenyls) Compounds

Bis(polyhydroxyphenyls) compounds or dicatechols of the present invention may be isolated from natural products such as botanical products, spices, oils and herbal extracts. For example, NDGA can be isolated from larrea divaricata and related plant species, and sesamin from sesame. Generally, “isolated” will refer to an organic molecule or group of similar molecules that have been subjected to fractionation to remove various other components, and which composition substantially retains its expressed biological activity. A “substantially purified” compound of the present invention refers to a composition in which bis(polyhydroxyphenyls) compound form the major component of the composition, such as constituting about 50%, about 60%, about 70%, about 80%, about 90%, about 95% or more of the molecules in the composition.

Extraction and purification techniques are well known to those of skill in the art. Following extraction and separation of the compounds of the present invention from natural products, purification techniques as described herein (for example, chromatographic techniques), may be used to achieve partial or complete purification (or purification to homogeneity).

Although numerous variations are possible, current general procedures for obtaining crude compounds typically include extraction with methanol, ethanol, water or aqueous alcohol; by a defatting step, generally with petroleum ether, performed before the extraction step or on the extract itself; by dissolution or suspension of the extract in water; by shaking or washing the solution or suspension with n-butanol saturated with water; and precipitation with such diethyl ether or acetone. Additionally, other techniques such as dialysis can also be included in order to remove small water-soluble molecules (Zhou et al., 1981; Massiot et al., 1988).

A variety of separation techniques have been described as is known to those of ordinary skill in the art, and may be used for separating bis(polyhydroxylphenyl) compounds including flash chromatography, low-pressure liquid chromatography (LPLC), medium-pressure liquid chromatography (MPLC), HPLC and conventional open-column chromatography (Hostettmann et al., 1986; Marston et al., 1991). Separation conditions, solvent systems, etc. will be known to those of skill in the art in light of the instant disclosure. The best results are usually achieved by strategies which employ a combination of methods.

Other methods that may also be employed in separating compounds, include but are not limited to dialysis, ion-exchange chromatography and size-exclusion chromatography. Distillation or supercritical extraction methods may also be employed. In some instances, compounds of the present invention can effectively be separated using organic solvents or solvent/water systems as are known to one of ordinary skill in the art.

A bis(polyhydroxylphenyl) compound may be isolated from other components, wherein the composition is purified to any degree relative to its naturally-obtainable state. In certain embodiments, it is contemplated that less substantially purified products of the present invention will have utility. Thus, partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of product, or in maintaining the biological activity of the bis(polyhydroxylphenyl) compounds.

In other embodiments the bis(polyhydroxyphenyl) compounds of the present invention may be chemically synthesized using conventional techniques as is known to one of ordinary skill in the art (Stewart and Young 1984; Tam et al., 1983; Merrifield 1986; and Barnay and Merrifield, 1979; each incorporated herein by reference). Bis(polyhydroxyphenyl) compounds of the present invention may also be chemically synthesized using a variety of techniques for symmetric synthesis as is known to one of ordinary skill in the art such as Witting condensation, or Schiff-base reactions.

V. Rational Drug Design of bis(polyhydroxyphenyl) Compounds

The bis(polyhydroxyphenyl) compounds of the present invention may be used in rational drug design to produce structural analogs of biologically active compounds. By creating such analogs, it is possible to fashion drugs which are more active or stable than the natural molecules, which have different susceptibility to alteration or which may affect the function of various other molecules. In one approach, one would generate a three-dimensional structure for the bis(polyhydroxyphenyl) compounds of the invention or a fragment thereof This could be accomplished by x-ray crystallography, computer modeling or by a combination of both approaches. An alternative approach, involves the random replacement of functional groups throughout the bis(polyhydroxyphenyl) compound, and the resulting affect on function determined.

It is also possible to isolate a bis(polyhydroxyphenyl) specific antibody, selected by a functional assay, and then solve its crystal structure. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

Thus, one may design drugs which have improved biological activity, for example, anti-inflammatory or anti-carcinogenic activity, relative to a starting bis(polyhydroxyphenyl) compound. By virtue of the chemical isolation procedures and descriptions herein, sufficient amounts of the bis(polyhydroxyphenyl) compounds of the invention can be produced to perform crystallographic studies. In addition, knowledge of the chemical characteristics of these compounds permits computer employed predictions of structure-function relationships.

VI. Pharmaceutical Formulations and Delivery of Bis(polyhydroxyphenyl) Compounds

Pharmaceutical compositions of the present invention comprise an effective amount of a bis(polyhydroxyphenyl) compound and optionally additional agent(s) dissolved or dispersed in a pharmaceutically acceptable carrier. The phrases “pharmaceutical or pharmacologically acceptable” refer to molecular entities and compositions that do not produce an adverse, allergic or other untoward reaction when administered to an animal, such as, for example, a human, as appropriate. The preparation of an pharmaceutical composition will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 1990, incorporated herein by reference). Except insofar as any conventional carrier is incompatible with the active ingredient, its use in the therapeutic or pharmaceutical compositions is contemplated.

The pharmaceutical composition may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intrapleurally, intranasally, topically, intratumorally, intramuscularly, subcutaneously, intraocularally, orally, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to a subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In certain embodiments, pharmaceutical compositions are prepared for administration by oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

VII. Combination Therapy

The compounds and methods of the present invention may be used in the context of neuroinflammatory diseases/conditions including but not limited to cancer or hyperplasia. In order to increase the effectiveness of a treatment with the compositions of the present invention, such as bis(polyhydroxyphenyls) and O-alkyl derivatives thereof, it may be desirable to combine these compositions with other agents effective in the treatment of those diseases and conditions. For example, the treatment of a cancer may be implemented with therapeutic compounds of the present invention and other anti-cancer therapies, such as anti-cancer agents or surgery. Likewise, the treatment of a neuroinflammatory disease or condition may involve bis(polyhydroxyphenyls) and O-alkyl derivatives thereof, of the present invention and conventional neurological agents or therapies. In other embodiments of the invention, non-bis(polyhydroxyphenyls) may be used in combination with bis(polyhydroxyphenyls) and O-alkyl derivatives thereof in treating neurological diseases.

Alternatively, other neurological diseases or conditions such as: amyotrophic lateral sclerosis (ALS) and other motor neuron diseases (MNDs) of similar clinical presentation; Parkinson's disease (PD); Alzheimer's disease (AD); spino-bulbar atrophy; (SBA); Huntington's disease (HD); myasthenia gravis (MG); multiple sclerosis (MS); HIV-associated dementia; fronto-temporal dementia (FTD); stroke; traumatic brain injury; age-related retinal degeneration; and encephalomyelitis, may be treated with compositions and by methods of the present invention in combination with therapeutic agents typically employed in the treatment of the particular neurological disease or condition.

Various combinations of times of treatment may be used in the present invention. For example, therapies involving bis(polyhydroxyphenyl) compounds may precede or follow that of other neuropharmaceutical agents by intervals ranging from minutes to weeks. Where other neuropharmaceutical agents, and bis(polyhydroxyphenyl) or O-alkyl derivative thereof are provided or administered separately to the subject, one would generally ensure that a significant period of time did not expire between the time of each delivery, such that the other neuropharmaceutical agent and bis(polyhydroxyphenyl) or O-alkyl derivative thereof would still be able to exert an advantageously combined effect on the subject. In such instances, it is contemplated that one may provide or administer to the subject both agents within about 1-6 or about 12-24 hr of each other and, more preferably, within about 6-12 hr of each other, with a delay time of only about 12 hr being most preferred. In some situations, it may be desirable to extend the time period for treatment significantly, however, where several days (2, 3, 4, 5, 6, or 7) to several weeks (1, 2, 3, 4, 5, 6, 7 or 8) lapse between the respective administrations.

It also is conceivable that more than one administration of either the bis(polyhydroxyphenyls) and/or O-alkyl derivatives thereof and/or secondary agent will be desired. Various combinations may be employed, where the bis(polyhydroxyphenyls) and O-alkyl derivatives is “A” and the secondary agent is “B”, as exemplified below:

A/B/A B/A/B B/B/A A/A/B B/A/A A/B/B B/B/B/A B/B/A/B A/A/B/B A/B/A/B A/B/B/A B/B/A/A B/A/B/A B/A/A/B B/B/B/A A/A/A/B B/A/A/A A/B/A/A A/A/B/A A/B/B/B B/A/B/B B/B/A/B

Administration of the bis(polyhydroxyphenyls) of the present invention to a subject will follow general protocols for the administration of that particular secondary therapy, taking into account the toxicity, if any, of the bis(polyhydroxyphenyl) compound treatment. It is expected that the treatment cycles would be repeated as necessary. It also is contemplated that various standard therapies, as well as surgical intervention, may be applied in combination with the described bis(polyhydroxyphenyl) compounds.

1. Antibiotics

Gentamicin—an antibiotic, may allow muscle cells to ignore an abnormal stop signal (premature stop codon) that tells the cell to stop making a needed protein too early in the production process. Gentamicin may be effective in stabilizing the muscle cell membrane. Other antibiotics which may be used with the present invention include, but are not limited to, amikacin, other aminoglycosides (e.g., gentamycin), amoxicillin, amphotericin B, ampicillin, antimonials, atovaquone sodium stibogluconate, azithromycin, capreomycin, cefotaxime, cefoxitin, ceftriaxone, chloramphenicol, clarithromycin, clindamycin, clofazimine, cycloserine, dapsone, doxycycline, ethambutol, ethionamide, fluconazole, fluoroquinolones, isoniazid, itraconazole, kanamycin, ketoconazole, minocycline, ofloxacin), para-aminosalicylic acid, pentamidine, polymixin definsins, prothionamide, pyrazinamide, pyrimethamine sulfadiazine, quinolones (e.g., ciprofloxacin), rifabutin, rifampin, sparfloxacin, streptomycin, sulfonamides, tetracyclines, thiacetazone, trimethaprim-sulfamethoxazole, viomycin or combinations thereof.

2. Anticancer Agents

The bis(polyhydroxyphenyl) compound(s) of the present invention may also be used in treating neuroinflammatory diseases, cancers or hyperplasias. Therefore, in specific embodiments, the present invention may be used in combination with other anti-cancer therapies which include biological agents (biotherapy), chemotherapy agents, and radiotherapy agents, as is known to those of skill in the art. An anti-cancer agent is capable of negatively affecting cancer in a subject. In the context of the present invention, it is contemplated that a bis(polyhydroxyphenyl) compound(s), can be used in conjunction with chemotherapeutic, radiotherapeutic, immunotherapeutic or other biological intervention, in addition to other pro-apoptotic or cell cycle regulating agents or surgery. Thus, it is contemplated that one or more anti-cancer therapies, as is known to one of ordinary skill in the art, may be employed with the bis(polyhydroxyphenyl) compound(s) as described herein. Some examples of anticancer agents include but are not limited to: 5-fluorouracil; α and γ interferon; mitotic inhibitors which include, for example, docetaxel, etoposide (VP16), teniposide, paclitaxel, methotrexate, taxol, vinblastine, vincristine, and vinorelbine; and any of the other combinations of therapies described herein.

3. Inhibitors of Cell Death/Promoters of Cell Survival

Inhibitors of cell death may also be used with the bis(polyhydroxyphenyls) of the present invention. These include but are not limited to the Bcl-2 family of proteins which promote cell survival such as Bcl-2, Bag, Bclₓₗ, Bcl_{w}, Bclₛ, Mcl-1, A1, and Bfl-1; NAIP (neuronal inhibitor of apoptosis) and IAPs (inhibitor of apoptosis) which appear to act by preventing the activity of caspases and/or their activation; promoters of cell survival such as NFκB; caspase inhibitors and calpain inhibitors, may all be more effective with the present invention in treating neurological diseases.

4. Other Inhibitors

Other inhibitors may also be used in conjunction with the bis(polyhydroxyphenyls) of the present invention. These may include but are not limited to protease inhibitor such as indinavir, which inhibit apoptosis of CD4 lymphocytes; inhibitors of caspases; cyclooxygenase-2 (COX-2) inhibitor which inhibits production of prostaglandins, that trigger astrocytic glutamate release and by inducing free radical formation. Beta-site APP (amyloid precursor protein)—BACE1 and BACE2 inhibitors; macrophage migratory inhibitor factor (MIF); phosphodiesterase inhibitors (PDEIs); inhibitors of nitric oxide; and macrophage migration inhibitory factor (MIF).

5. Neurotrophic Factors/Genes

Neurotrophic factors are essential for the growth, maturation and survival of nerve cells and may be used in combination with the present invention in treating neurological diseases/conditions. These include but are not limited to: CNTF—ciliary neurotrophic factor; NT3—neurotrophic factor 3; BDNF—brain derived neurotrophic factor; GNDF—glial cell derived neurotrophic factor; NGF—nerve growth factor; and other neurotrophic factors such as—Insulin-like Growth Factor-1 (IGF-1; Myotrophin®) which is a essential for normal development of the nervous system. Additionally, purine derivatives, a class of drug compounds which includes neotrofin(™) (AIT-082, leteprinim potassium), and can be used to selectively control “turning on or off of genes” involved in nerve regeneration, may also be used with the bis(polyhydroxyphenyls) of the present invention. Compounds that possesses neurotrophin-like activity such as xaliproden—a nonpeptide may also be used.

6. Non-Steroidal Anti-Inflammatory Drugs (NSAIDs)

It is also contemplated by the present invention that non-steroidal anti-inflammatory drugs (NSAIDs) may also be used in combination. These include but are not limited to: naproxen; indomethacin; ibuprofen; fenoprofen; diclofenac potassium, diclofenac sodium, diclofenac sodium with misoprostol, diflunisal, etodolac, fenoprofen calcium, flurbiprofen, ketoprofen, meclofenamate sodium, mefenamic acid, meloxicam, nabumetone, oxaprozin, piroxicam, sulindac, tolmetin sodium; cox-2 inhibitors: celecoxib, rofecoxib; salicylates—acetylated: aspirin; non-acetylated: choline salicylate, choline and magnesium salicylates, salsalate, and sodium salicylate.

7. Steroids

Steroids are also contemplated for use in combination with the bis(polyhydroxyphenyls) of the present invention. These include but are not limited to: corticosteroids; methylprednisolone; baclofen (Lioresal®), tizanidine (Zanaflex®) and the benzodiazepines, such as diazepam (Valium®;) prednisone, dexamethasone, hydroxychloroquine (Plaquenil), azathioprine (Imuran), mycophenolate mofetil (Cell Cept), methotrexate, or cyclophosphamide (Cytoxan).

8. Immune System Therapy

Treatment for some neurological diseases can be directed at modulating or changing the response that the immune system directs toward the central nervous system. Such therapeutic modalities may also be used with the bis(polyhydroxyphenyls) of the present invention. These include but are not limited to: interferons (INFs) which occur naturally in our immune system and may be helpful in limiting inflammation and further include IFN-β1b (Betaseron®); IFN-β1a (Avonex®);and IFN-β1-a (Rebif®). Additionally, glatiramer acetate which modifies some of the actions of the immune system that is thought to play a role in the progression of certain neurological diseases may also be used in combination with the present invention.

9. Glutamate Therapy

It is also contemplated by the present invention that agents or therapies that reduce, suppress, inhibit or regulate glutamate levels, of which excess is toxic to neurons, may be used with the bis(polyhydroxyphenyls) of the present invention. These include but are not limited to: Tamoxifen—a protein kinase C inhibitor that could produce an anti-glutamate effect; Rilutek®—a glutamate-blocking drug used in ALS therapy; NAALADase inhibition (NAAG (N-Acetyl-Aspartyl-Glutamate) is converted by NAALADase (N-Acetylated-Alpha-Linked-Acidic-Dipeptidase) into glutamate); NMDA antagonists such as memantine and nitroglycerin, and the combination drug nitro-memantine, may also be used.

10. Antioxidants

Antioxidants may also be employed in the present invention as a combination therapy with the bis(polyhydroxyphenlys) of the invention in treating inflammatory diseases. Antioxidants may include but are not limited to, methionine, choline, N-acetylcysteine, vitamins (e.g., B complex—vitamin B₆ or vitamin B₁₂; vitamin K; vitamin E—tocopherols; vitamin A; vitamin C; and derivatives thereof), gluthathione, cysteine, and 2-mercaptoethanol, idebenone, co-enzyme Q10, ALA, camosine, tocotrienols, flavonoids, ALC, probucol, ascorbic acid, vinpocetine, lipoic acid, carotenoids, selenium, lycopene, creatine, arginine, taurine, cysteine, nicotinamide adenine dinucleotide, resveratrol, ginkgo biloba, oligomeric proanthocyanidins, and phenolic antioxidants.

11. Other Therapy

Other agents or therapies that may be used with the bis(polyhydroxyphenyls) of the present invention include: transplantation of embryonic cells such as embryonic dopamine cells; folic acid treatments; telomerase therapy; and agents such as creatine and albuterol.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example I

ALS Involves Neuroinflammatory Events Indicative of Microglial Activation, Especially Increased Expression of TNFα and the TNF Receptor I

The G93A-SOD1 mutant mouse: A model of motor neuron disease associated with oxidative damage to the CNS. Approximately 20% of all inherited cases of ALS are caused by mutations in the antioxidant enzyme Cu, Zn-superoxide dismutase (SOD1); one of the most common mutations is a glycine to alanine substitution at residue 93 of the enzyme (G93A-SOD1) (Rosen et al., 1993; Deng et al., 1993). A colony of G93A-SOD1 transgenic mice, the standard model for ALS, was established (Rosen et al., 1993; Deng et al., 1993; Hall et al., 1998). Animals bearing the mutant transgene experience spinal column degeneration beginning at 90-100 days of age. Animals were killed when no longer able to right themselves within 30 sec of being placed on their sides.

Temporal Correlation Between Spinal Cord Degeneration in the G93A-SOD1 Mouse and Expression of Inflammatory Cytokines, Particularly TNFα and TNFR-I

Multiprobe ribonuclease protection assays (RPAs) have been used extensively to index inflammation and apoptosis during periods of pathophysiological stress (Gabbita et al., 2000; Stewart et al., 1999). RPAs allow the simultaneous quantitation of multiple mRNA species with 10-fold greater sensitivity than Northern blots. RPAs indicated a macrophage-typical (monokine) pattern of cytokine expression in G93A-SOD1 mouse spinal cords at latter stages of life. Several (but not all) monokines were significantly elevated in the G93A-SOD1 mouse spinal cord at 120 days of age. For instance, interleukin 1α (IL1α) and IL1β were robustly increased in 120-day old G93A-SOD1 mice relative to nontransgenic littermates or to mice expressing wildtype human SOD1 (Table II). Others have very recently reported elevated TNFα in presymptomatic and afflicted G93A-SOD1 mouse cord (Yoshihara et al., 2002). Interestingly, the IL1 receptor antagonist (IL1RA) was comparably increased (Table II) indicating that some anti-inflammatory components are also upregulated in the G93A-SOD1 mouse spinal cord. The strongly anti-inflammatory IL10, which can be generated by macrophages or T-cells, was not significantly affected at 120 days (Table II). Subsequently, RPAs were used to assess the same cytokine mRNAs in spinal cord of 80 day-old animals, before onset of paralysis. These data indicate that monokine up-regulation (indicating microglial activation) precedes and correlates with onset of paralysis in the G93A-SOD1 mouse (Table II). Contrastingly, T-lymphocyte derived cytokines (lymphokines) such as IFNγ, IL2, IL3, IL4, IL5 and IL15 were expressed at lower levels and these were only marginally altered in G93A-SOD1 mice.

Separate RPAs were performed to assess expression of apoptosis-associated genes at 120 D and 80 days, representing symptomatic and late pre-symptomatic periods respectively (Table II). All of the caspase mRNAs were increased at 120 D, but none were significantly increased at 80 D. Likewise, specific “death receptors” such as FAS were unchanged or only slightly increased at 80 days but were strongly elevated by 120 days of age (Table II). The notable exception is TNFR-p55 (TNF-RI), which was significantly elevated at 80 D and increased further at 120 D. Thus, up-regulation of pro-apoptotic genes follows cytokine changes but temporally correlates with onset of total hindlimb paralysis. These data indicate that the TNFα/TNF-RI system is especially important to the pathogenesis of ALS.

TABLE II
Summary of RPA Data

| | 80 D % | 120 D | |
|---|---|---|---|
| | change relative to | % change relative | |
| | nonTg mice | wt-SOD mice | nonTM mice |
| Cytokines | | | |
| TNFα | 152* | | 717* |
| IL1α | 217 ± 22* | 397 ± 10 | 294 ± 17* |
| IL1β | 178 ± 18* | 760 ± 27* | 183 ± 18* |
| I L1 RA | 355 ± 20* | 2085 ± 13* | 415 ± 14* |
| I1-6 | ND | 155 ± 5* | ND |
| IL10(M) | 133 ± 21 | 117 ± 6 | 127 ± 18 |
| IL10(L) | 115 ± 6 | | 93 ± 7 |
| IL12-p35 | 235 ± 15* | 135 ± 8* | 131 ± 19 |
| IL18 | 158 ± 20* | 99 ± 3 | 107 ± 7 |
| MIF | 116 ± 3* | 73 ± 3* | 89 ± 7 |
| I FNγ(M) | 112 ± 5 | 122 ± 4* | 119 ± 8 |
| IFNγ(L) | 118 ± 12 | | ND |
| IL2 | ND | | 90 ± 7 |
| IL3 | ND | | 99 ± 9 |
| IL4 | ND | | 123 ± 9 |
| IL5 | 118 ± 22 | | 95 ± 7 |
| IL15 | 118 ± 6 | | 80 ± 6 |
| Caspases | | | |
| caspase 1 | 91 ± 13 | 290 ± 6* | 275 ± 14* |
| caspase 2 | 104 ± 5 | 136 ± 3* | 106 ± 12 |
| caspase 3 | 104 ± 5 | 207 ± 2* | 120 ± 20 |
| caspase 6 | 101 ± 8 | 228 ± 3* | 145 ± 18* |
| caspase 7 | 112 ± 18 | 241 ± 5* | 147 ± 9* |
| caspase 8 | 101 ± 12 | 420 ± 4* | 204 ± 21* |
| caspase 11 | 88 ± 25 | 461 ± 6* | 228 ± 7* |
| caspase 12 | 99 ± 16 | 668 ± *6 | 286 ± 8* |
| Death Receptors | | | |
| Fas | 66 ± 16 | 269 ± 6* | 420 ± 4* |
| FasL | 90 ± 10 | 95 ± 11 | 147 ± 6* |
| FADD | 150 ± 8* | 145 ± 5 | 119 ± 3 |
| FAF | 118 ± 7 | 109 ± 9 | 82 ± 2 |
| FAP | 136 ± 13 | 92 ± 7 | 90 ± 2 |
| TNFR-p55 | 64 ± 6* | 357 ± 5* | 333 ± 5* |
| TRAIL | 51 ± 17* | 100 ± 7 | 92 ± 4 |
| TRADD | 107 ± 4 | 100 ± 3 | ND |
| RIP | 115 ± 6 | 173 ± 7* | 174 ± 6* |

Values obtained from G93A-SOD1 mice are expressed as a relative percentage to the mean value (± SEM) obtained from nontransgenic (NonTg) or wt-SOD1 mice at each age. *p < 0.05 by individual t-tests. ND = below detection limits. (M) Denotes value obtained from monokine probe set; (L) denotes value from lymphokine probe set.

### Example II

Efficacy of Tethered Bis(Polyhydroxyphenyl) Compounds Relative to Other Linked Bis(Polyhydroxyphenyl) Compounds and to Non-Steroidal Anti-Inflammatory (NSAID) Agents

Reactive oxygen species (ROS) and reactive nitrogen species (RNS) have been strongly associated with ALS, as described previously. Microglia and macrophages stimulated with pro-inflammatory agents such as bacterial lipopolysaccharide (LPS) generate O₂.⁻ and .NO (Colton et al., 1994; Meda et al., 1995). For these studies EOC-20 mouse microglial cells were, selected as the model system. EOC-20 cells are a characterized, non virus-transformed, CSF-1 dependent mouse microglial cell line that expresses IgG receptors FcγRI and II; Mac-1, Mac-2, Mac-3, CD45, CD80 and MHC-I constitutively and expresses MHC-II in response to IFNγ (Walker et al., 1995). EOC-20 cells therefore closely resemble macrophages and primary microglial in so far as they have been characterized.

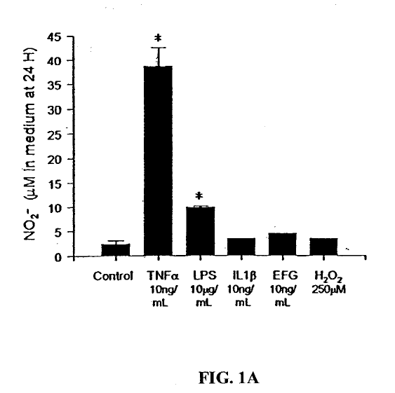

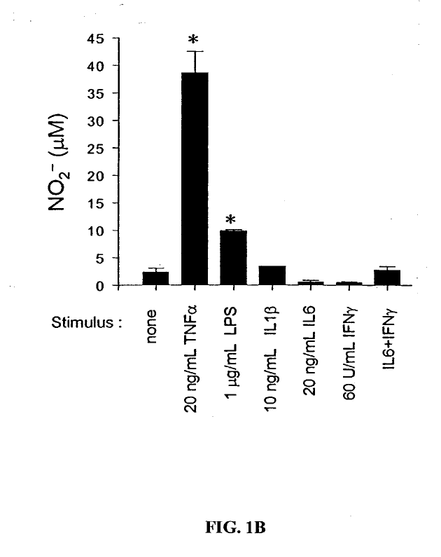

As shown in FIG. 1, EOC-20 cells stimulated with TNFα produce robust amounts of RNS as evidenced by nitrite (NO₂⁻) accumulation in the medium. Nitrite is an autoxidation product of .NO (Shishido et al., 2001; Yijanheikki et al., 1999) that can be measured using a convenient colorimetric Griess diazotization assay (Marzinzig et al., 1997; Archer, 1993). In macrophage-type cells stimulated with archetypal inflammatory agents, NO₂⁻ reflects mostly iNOS expression (Colton et al., 1994; Meda et al., 1995). FIG. 1 demonstrates that TNFα is a potent stimulus of RNS in EOC-20 cells, causing them to generate more NO₂⁻ than other archetypal stimuli such as bacterial lipopolysaccharide (LPS). Interestingly, the TNFα effect is somewhat specific in that EOC-20 cells fail to generate nitrite in response to IL1β, EGF or H₂O₂ (FIG. 1). Heat-aggregated, but not free, IgG will also stimulate these cells modestly, with approximately the same potency as LPS.

After establishing the microglial response to TNFα in vitro, microglial cell cultures were stimulated with TNFα in the presence of natural product components of “neutriceuticals” that have been studied recently for anti-proliferative or anti-inflammatory action. Cells were cultured in 24 well plates until confluent then treated with test agent, which was dissolved at 100× final concentration in dimethylsulfoxide (DMSO), or with vehicle only. Thirty minutes after treatment with test agent, cells were treated with 10 ng/mL recombinant mouse TNFα (Pharmingen, San Diego, Calif. USA). After 24 hr, 0.1 mL aliquots were withdrawn from the culture medium for purposes of assaying NO₂⁻ using commercially available reagents (Marzinzig et al., 1997; Archer, 1993). The optical density of the resulting diazo chromophore was measured at 560 nm in a microplate format using authentic nitrite as an external standard. Each compound was tested at 100 μM, 20 μM, 4 μM, 0.8 μM and 0 μM in the presence of TNFα. Four wells of cells were used for each concentration of test agent.

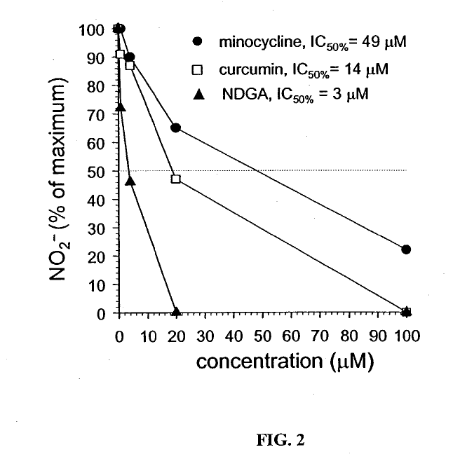

For each test compound, an IC₅₀ value was determined by interpolation; this value represents the concentration needed to inhibit nitrite production by 50%. Lower IC₅₀ values indicate improved efficacy. FIG. 2 illustrates the principle of the measurement using NDGA as an example test agent. In order to determine viability, cell culture medium was replaced with phenol-free DMEM (Gibco) containing 20 μL/mL of MTT viability reagent (Promega). After 1 hr incubation at 37° C., 200 μL of the medium was removed and optical density measured at 540 nm in a microplate format using diluted MTT viability reagent (without cells) as a blank (100% toxicity). Control cells were taken to indicate 100% viability and intermediate values of MTT reduction were scaled to percentage of control. For each compound, an LD₅₀ value was determined by extrapolation through sublethal concentrations. This value represents the concentration that reduced the MTT viability parameter by 50%.

Table III summarizes the results of a survey of natural and synthetic products including both tethered bis(polyhydroxyphenyl) compounds and some agents that are linked (but not tethered) bis(polyhydroxyphenyl) compounds. It is clear from this comparison that tethered bis(polyhydroxyphenyl) compounds are generally more potent than those bis(polyhydroxyphenyl) compounds that are merely linked but not tethered. It is also clear that efficacy generally correlates with the number of hydroxyl groups on rings A and B. For example piceatannol, which has two hydroxyl groups on each ring, is more active than resveratrol, which has one hydroxyl group on one ring and two hydroxyl groups on the second ring. Increasing the length of the tether between rings A and B may increase efficacy, since NDGA is more effective than piceatannol.

The benchmark lipoxygenase inhibitor caffeic acid phenethyl ester (CAPE), which inhibits lipoxygenase almost 10-times more effectively than does NDGA (Mirzoeva and Calder, 1996), is 4-fold less effective as an inhibitor of TNFα-stimulated microglial activation. Moreover, CAPE is structurally related to the bis(polyhydroxyphenyls) in that CAPE is a tethered bisphenyl compound, but CAPE fails to satisfy the definition of a tethered bis(polyhydroxyphenyl) because CAPE lacks the required hydroxylation (or alkoxylation) of both phenyl rings (Mirzoeva and Calder, 1996). Thus CAPE might coincidentally display some activity as a C-RTK inhibitor in addition to inhibiting lipoxygenase. These data indicate that the novel action of highly effective bis(polyhydroxyphenyl) compounds is not exclusively due to lipoxygenase inhibiting action.

Table III also specifically includes the tetra-ethyl (ethyloxy) ether NDGA (Et₄NDGA), which was synthesized by the inventors using a modification of published methylation techniques (Hwu et al., 1998). The data regarding Et₄NDGA demonstrate that substituting ethyl ethers (an alkoxyl group) for the hydroxyl groups can further increase potency although, in this specific example, at the cost of some cytotoxicity (Table III). This data indicates that certain O-alkyl derivatives of tethered bis(polyhydroxyphenyl) compounds can be superior inhibitors of microglial activation. Furthermore it is clear that most tested members of the class of tethered bis(polyhydroxyphenyl) compounds shown in Table III, including piceatannol and NDGA, are more effective than benchmark NSAIDs such as indomethacin and ibuprofen, when evaluated against the same microglial activation assay (Table III). Finally it is clear that most tested members of the class of tethered bis(polyhydroxyphenyl) compounds are more potent than the benchmark microglial-inhibiting agent minocycline (Yijanheikki et al., 1999), when evaluated against the same microglial activation assay (Table III).

Finally, Table III demonstrates the superiority of many tethered bis(polyhydroxyphenyl) compounds relative to benchmark therapeutics riluzole and minocycline. In general the tethered bis(polyhydroxyphenyl) derivatives inhibit microglial activation at lower doses than riluzole or minocycline, with lesser cytotoxicity. For example, rooperol had an IC₅₀ of 25 μM and was nontoxic. Riluzole is currently the only U.S.-approved compound for treating ALS (Physician's Desk Ref. 2002) while minocycline is being investigated for treating ALS, HD and PD (Chen et al., 2000; Zhu et al, 2002; Wu et al., 2002). Thus, riluzole can be considered a clinical benchmark. Some tethered bispolyhydroxyphenyls), such as NDGA, are much less toxic than riluzole in vivo. For example, the LD₅₀ for riluzole (oral route) is 94 mg/kg in mice and 40 mg/kg in rats ((Physician's Desk Ref. 2002) whereas for NDGA, the LD₅₀ is greater than 500 mg/kg i.p. in mice and more than 2000 mg/kg oral in rats (Lehman et al., 1951). Mice fed 0.5% of the diet as NDGA long-term show no ill effects (Cranston et al., 1947). Likewise, NDGA may be less toxic than minocycline, which can cause photsensitization, liver damage, thyroid and lingual discoloration, and lupus-like disease (Physician's Desk Ref. 2002; Balestrero et al., 2001). NDGA also is more cost-effective to synthesize or purify than riluzole and minocycline. For instance, commercial preparations of NDGA cost approximately $46/g while riluzole costs $800/g and minocycline costs $180/g (supplied by Sigma Chemical, St. Louis Mo.).

TABLE III
Relative Efficacies of Select Compounds Tested
Against TNFα-Stimulated NO₂⁻ Production in EOC-20 Cells.

| | | | | Class and |
|---|---|---|---|---|
| | | | | Presumed Function |
| | | | | T = tethered |
| | | | | or L = linked |
| | IC₅₀ | LD₅₀ | LD₅₀/ | bis(polyhydroxy- |
| Name | μm | μm | IC₅₀ | phenyl); O = other |
| Flavone | 60 | 7 | 1.27 | Flavonoid (L) |
| 3-OH-flavone | 70 | 125 | 1.79 | Flavonoid (L) |
| 5-OH-flavone- | 34 | 310 | 9.12 | Flavonoid (L) |
| nitro-5-OH-flavone | 81 | 254 | 3.14 | Flavonoid (L) |
| 7-OH- flavone | 65 | 125 | 1.92 | Flavonoid (L) |
| 5,7-dihydroxy-flavone | 63 | 85 | 1.35 | Flavonoid (L) |
| (chrysin) | | | | |
| nitro-chrysin | Inactive | nontoxic | | Flavonoid (L |
| 3,5,7-trihydroxy- | 41 | 58 | 1.41 | Flavonoid (L) |
| flavone (galangin) | | | | |
| 4′,5,7-trihydroxy- | 55 | 87 | 1.58 | Flavonoid (L) |
| flavone (apigenin) | | | | |
| 3,4′,5,7-tetrahydroxy- | 47 | 70 | 1.49 | Flavonoid (L) |
| flavone (kaempherol) | | | | |
| 3,3′,4′,5,7-tetra- | 44 | nontoxic | | Flavonoid (L) |
| hydroxy-flavone | | | | |
| (quercetin) | | | | |
| Chrysene | Inactive | nontoxic | | Flavonoid (L) |
| 4,7-dihydroxyiso- | 114 | nontoxic | | Flavonoid (L) |
| flavone (daidzein) | | | | |
| 4-OH-4′-methoxy-iso- | 106 | 126 | 1.19 | Flavonoid (L) |
| flavone (formononetin) | | | | |
| 4′-methoxy-5,7- | 49 | 112 | 2.29 | Flavonoid (L) |
| dihydroxyisoflavone | | | | |
| (biochanin A) | | | | |
| 4′,5,7-trihydroxy- | 46 | nontoxic | | Flavonoid (L) |
| isoflavone (genistein) | | | | |
| 4′,5-dihydroxy-7- | 120 | nontoxic | | Flavonoid (L) |
| methoxy-isoflavone | | | | |
| (prunetin) | | | | |
| Trans- stilbene | Inactive | nontoxic | | Stilbenoid (O) |
| Rhapontin (natural, | Inactive | nontoxic | | Stilbeneoid |
| rhubarb) | | | | glycoside (O) |
| Ellagic acid | 110 | nontoxic | | Hydroxy-bisphenyl |
| | | | | (O) |
| Tyrphostin 51 | Inactive | nontoxic | | Tyrphostin |
| | | | | optimized for |
| | | | | EGF-RTK (O) |
| Tyrphostin AG126 | 18 | 150 | 8.33 | Tyrphostin |
| | | | | optimized against |
| | | | | LPS (O) |
| phenyl N tert-butyl- | 100 | nontoxic | | Nitrone (O) |
| nitrone (PBN) | | | | |
| 2-OH-PBN | Inactive | nontoxic | | Nitrone (O) |
| 3-OH-PBN | Inactive | nontoxic | | Nitrone (O) |
| 4-OH-PBN | Inactive | nontoxic | | Nitrone (O) |
| Tetracycline | 85 | nontoxic | | Tetracycline analog |
| | | | | (O) |
| Oxyletracycline | 64 | nontoxic | | Tetracycline |
| | | | | analog (O) |
| Minocycline | 49 | nontoxic | | Tetracyclin analog/ |
| | | | | microglial inhibitor |
| | | | | (O) |
| alpha tocopherol | Inactive | nontoxic | | Antioxidant (O) |
| Lipoic acid | Inactive | nontoxic | | Antioxidant (O) |
| pyrollidine dithio- | 51 | 90 | 1.76 | Antioxidant/NFkB |
| carbamate (PDTC) | | | | inhibitor (O) |
| Indomethacin | 61 | 124 | 2.03 | NSAID/ |
| | | | | COXinbibitor (O) |
| Ibuprofen | Inactive | nontoxic | | NSAID/ |
| | | | | COXinhibitor (O) |
| gingkolide A | Inactive | nontoxic | | Botanical |
| | | | | (unspecified action) |
| | | | | (O) |
| gingkolide B | Inactive | nontoxic | | Botanical |
| | | | | (unspecified action) |
| | | | | (O) |
| caffeic acid phenethyl | 12 | nontoxic | | Benchmark lipoxy- |
| ester (CAPE) | | | | genase inhibitor (O) |
| SB203580 | 18 | 100 | 5.56 | Inhibitor of p38 |
| | | | | MAP kinase (O) |
| Minocycline | 49 | nontoxic | | Proposed for use in |
| | | | | ALS, HD and PD |
| Riluzole | 95 | nontoxic | | Currently approved |
| | | | | for ALS |
| coenzyme O | Inactive | nontoxic | | Investigational drug |
| | | | | for ALS, HD |
| 3,4′,5-trihydroxy- | 80 | nontoxic | | Stilbenoid (T) |
| stilbene (resveratrol) | | | | |
| 3,3′,4′,5-tetrahydroxy- | 15 | nontoxic | | Stilbenoid (T) |
| stilbene (piceatannol) | | | | |
| nordihydroguaiaretic | 3.1 | nontoxic | | Bis(polyhydroxy- |
| acid (NDGA, natural) | | | | phenyl (T) |
| NDGA (synthetic) | 4 | nontoxic | | Bis(polyhydroxy- |
| | | | | phenyl (T |
| Tetra-ethyl ether of | 0.8 | 7.2 | 9.00 | Bis(polyhydroxy- |
| NDGA (synthetic) | | | | phenyl (T) |
| curcumin (natural) | 7 | 120 | 17.14 | Bis(polyhydroxy- |
| | | | | phenyl (T) |
| butein (2′,4′,3,4- | 5.1 | 77 | 15.10 | Bis(polyhydroxy- |
| tetrahydroxychalcone) | | | | phenyl (T) |
| Rosemarinic acid | 139 | nontoxic | | Bis(polyhydroxy- |
| | | | | phenyl (T) |
| bis(tyrphostin) | 100 | nontoxic | | Bis(polyhydroxy- |
| | | | | phenyl (T) |

### Example III

Tethered Bis(polyhydroxyphenyl) Compounds Suppress Transcription of Pro-Inflammatory Cytokines in Microglial Cells

A multiprobe ribonuclease protection assay (RPA) indicated that TNFα stimulated expression of specific cytokines in EOC-20 microglial cells. Piceatannol differentially inhibited transcription of the pro-inflammatory cytokines, though the inhibitory potency was somewhat less than observed for inhibition of RNS flux (Table III). L32 and GAPDH “housekeeping” gene products were included in the RPA probe set to demonstrate equivalency of sample loading across the several lanes. It was noted that IL1β, IL1RA, IFNγ and MIF were affected by the treatments, while IL18 was not.

### Example IV

The Tethered Bis(polyhydroxyphenyl) Compound NDGA Improves Prognosis of Motor Functional Decline in a Mouse Model of Amyotrophic Lateral Sclerosis (ALS)

A colony of G93A-SOD1 transgenic mice, the standard model for ALS, was established (Gurney et al., 1996; Johnson et al., 2000). These animals contain the mutant human superoxide dismutase (SOD1) enzyme responsible for a major subset of cases of familial amyotrophic lateral sclerosis (Rosen et al., 1993). Animals bearing the mutant transgene experience spinal column degeneration beginning at 90-100 days of age. Animals are killed when no longer able to right themselves within 30 sec of being placed on their sides. As a precedent for validity of this animal model, riluzole (the only drug currently approved to treat ALS, which delays progression of the disease only marginally in humans) causes a 10 day extension in lifespan in G93A-SOD1 mice when administered chronically (Gurney et al., 1996).

More subtle defects in motor performance are measured using a rotorod device. The animals are placed on a horizontal rod set to spin about its long axis, initially at 1 rpm. The revolution rate is increased at a constant rate of 1 rpm every 10 sec and the experiment continues until the animal falls off the rod. In contrast to mice bearing mutant SOD1 transgenes, animals bearing the wild-type human SOD1 transgenes display healthy motor function. Likewise, nontransgenic littermates demonstrate normal function.

The G93A-SOD1 mice were used to test for in vivo efficacy of a benchmark tethered bis(polyhydroxyphenyl) compound, nordihydroguaiaretic acid (NDGA). In one experiment, 10 G93A-SOD1 animals were trained to the rotorod task at 85 days of age. At 90 days of age, animals were divided into two groups of 5 animals each. One group was injected intraperitoneally with NDGA (10 mg/kg, 5 days/week, in 95% saline/5% DMSO vehicle). The control group received vehicle only. Animals were tested on the rotorod device every 5 days subsequently.

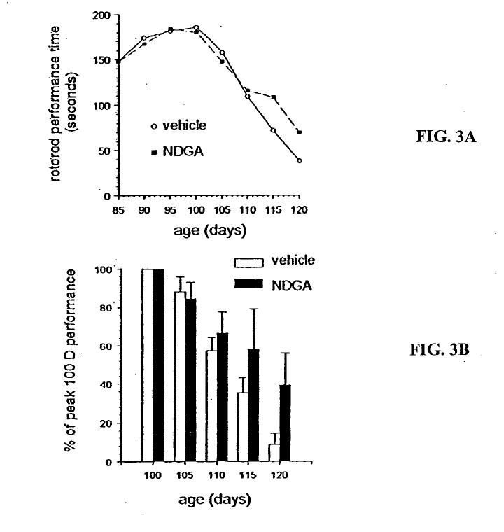

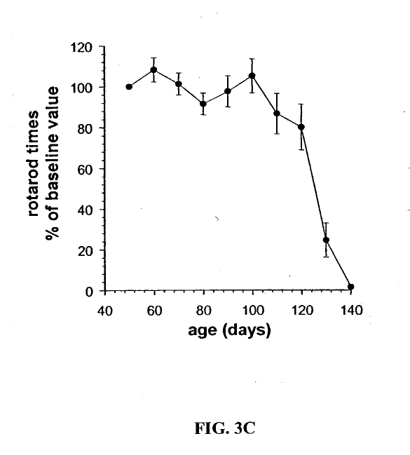

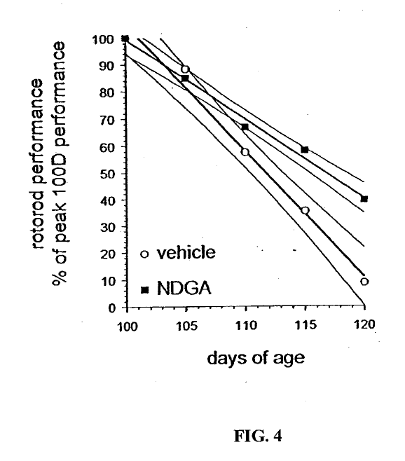

Initially, rotorod performance characteristics improve in all mice as the animals become acquainted with the apparatus. At 100 days animals obtain peak performance (FIG. 3A). At timepoints subsequent to 100 day, animals decline in motor performance. In order to control for differences in initial strength amongst the 10 animals, all data subsequent to the 100 day timepoint was expressed as a percentage relative to the peak performance at 100 day (FIGS. 3B and 3C). NDGA treatment caused a decrease in the rate of motor functional decline relative to animals receiving vehicle only. When the data in FIG. 3B were assessed using repeated measures analysis of variance (ANOVA), a significant age X treatment effect was observed (p<0.005). At the 120D timepoint, the statistical assessment by Student's t-test indicates p<0.08 between drug and vehicle groups (i.e. 92% probability of a protective drug effect). Linear regression through the five timepoints between 100-120D indicated a 38% slower rate of motor functional deterioration amongst NDGA treated animals relative to vehicle-treated animals (slope of vehicle-treated animals=4.71% decrease/day; slope of NDGA-treated animals=2.96% decrease/day; p<0.05; FIG. 4). An assessment of the prior art indicates that NDGA is the first compound to display any efficacy in the G93A-SOD1 mouse when administered systemically at a symptomatic stage of the disease.

### Example V

Effects of Bis(polyhydroxyphenyls) on LnCAP Production of PSA LnCAP Cells

LnCAP prostate cancer cells were be cultured according to established methods (Horoszewicz et al., 1983). These cells product prostate-specific antigen (PSA), a serine protease that predicts metastatic potential (Thalmann et al., 2000). Cells were treated with 0, 4 μM, 20 μM or 100 μM of drug (in DMSO vehicle) for 24 h. Each drug was tested in triplicate at each concentration. Medium was removed and tested for PSA concentration using a commercially available enzyme-linked immunosorbent assay (ELISA). Viability was determined in the adherent cells using the MTT assay.

TABLE IV
Drug Effects on LnCAP Production of PSA: IC₅₀ Values (ELISA)
Linked but not tethered compounds

| | |
|---|---|
| 5-OH-flavone: | 227 μM (extrapolated) |
| Chrysin: | 91 μM |
| Daidzein: | 130 μM (extrapolated) |
| Quercetin: | 131 μM (extrapolated) |
| Epicatechin: | Inactive |
| Kaempherol: | 68 μM |

Tethered bis(polyhydroxyphenyls)

| | |
|---|---|
| Piceatannol: | 42 μM |
| Resveratrol: | 43 μM |
| NDGA: | 15 μM |

Tethered bis(polyhydroxyphenyl) compounds such as piceatannol, resveratrol and NDGA were more effective on LnCAP production of PSA than linked bis(polyhydroxyphenyls) with an IC₅₀ of 42 μM, 43 μM and 15 μM respectively.

### Example VI

Sesamin Formulation (Sesame Oil) Improves Function in a Mouse Model for Amyotrophic Lateral Sclerosis (ALS)

In sesame oil, lignans carrying a hydroxy group, for example, sesaminol, episesaminol, and sesamolinol, exhibit antioxidant activity (Osawa et al., 1985; Fukuda et al., 1985); however, sesamin as an antioxidant has not been evaluated clearly. Additionally, the metabolized dicatechol products of sesamin in the liver of rats has been demonstrated (Nakai et al. (2003). However, the anti-inflammatory action)(s) of metabolized dicatechol products of sesamin are not known.

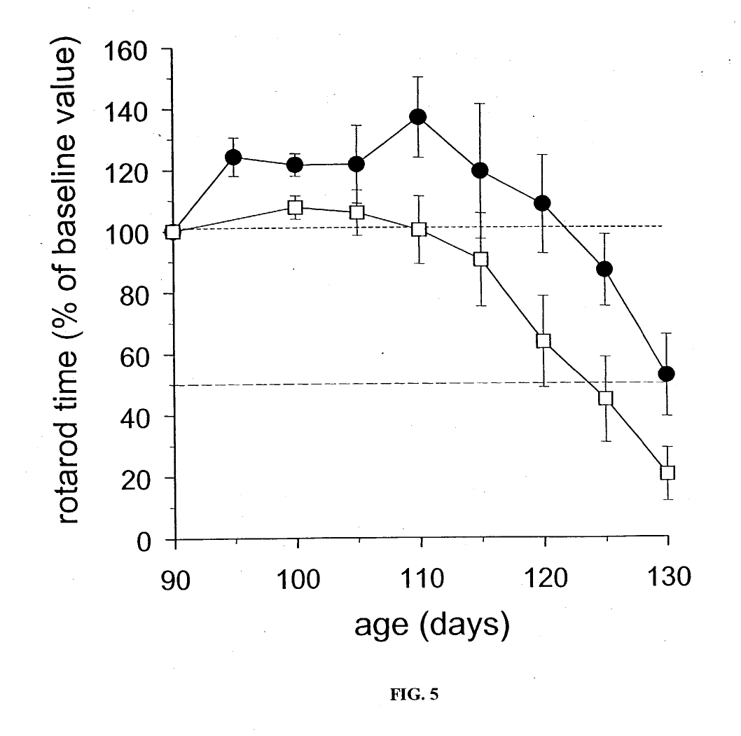

To determine the involvement of sesamin formulation in motor function, G93A-SOD1 mice were injected intraperitoneally (I.P.) with 100 μL of sesame oil each day, 5 days per week beginning at 90 D of age. Animals were rotarod tested at 90 D and at 5 day intervals thereafter. Animals were tested in quadruplicate and rotarod performance times were normalized to the baseline (90 D) performance time for each animal. ALS-afflicted animals receiving sesame injections performed significantly better than had been previously observed for untreated G93A-SOD1 mice (FIG. 5). In fact, performance increased in the sesame oil injected animals, up to approximately 115 D of age (FIG. 5).

### Example VII

Inhibitors of Arachidonic Acid Metabolism are Potent Antagonists of TNFα Signaling

Inhibitors of arachidonic acid metabolism, especially 5LOX inhibitors, are potent antagonists of TNFα signaling. EOC-20 cells present several attractive features that recommend their use as a bioassay for screening pharmacological agents for microglial-suppressing activity. EOC-20 cells are extremely easy to culture and grow very rapidly; they are rather robust with respect to surviving cell stress; they are readily stimulated by archetypal pro-inflammatory agents such as TNFα; and they produce a small molecule, NO₂⁻, that can be readily assayed in cell culture medium at the same time that viability is assessed.

In order to begin evaluating EOC-20 cells as a candidate for high-throughput screening, cell cultures were stimulated with TNFα in the presence of natural compounds or synthetic drugs that have been studied recently for anti-proliferative or anti-inflammatory action.

Three hundred structurally distinct and rationally chosen compounds have been tested in this cell culture system. Typical dose-inhibition profiles for NDGA, curcumin, and minocycline are illustrated in FIG. 2. The most noteworthy finding from this analysis was the striking potency of certain inhibitors of arachidonic acid metabolism as TNFα antagonists (Table V). In particular, the natural dicatechols curcumin and NDGA were both very effective, nontoxic inhibitors (Table V; FIG. 2). Both compounds were significantly more potent than the benchmark microglia inhibitor minocycline, which suppresses microglial responses in animal models of ALS, Huntington's disease and Parkinson's disease (Wu et al, 2002; Chen et al., 2000; Tikka et al., 2001; Zhu et al., 2002; Balestrero et al., 2001). In particular, NDGA was approximately 16 times more potent than minocycline, with an IC₅₀ value of 3-5 μM and no toxicity at 100 μM (FIG. 2). In terms of relative potency, this placed NDGA in the top 2% of nontoxic compounds tested. Significant NO₂⁻ suppression was observed at 800 nM NDGA. Similar efficacy was observed for natural and synthetic NDGA, as well as for the acetyl ester of NDGA (Table V). Interestingly, tetra-O-methyl NDGA (which does not inhibit lipoxygenase; Bensimon et al., 2002) displayed modest bioactivity, though it was less potent than the parent compound (Table V). The clinically approved 5LOX inhibitor, zileuton (Zyflo), was approximately as effective as minocycline but less potent than NDGA or curcumin (Table V). Weak to negligible activity was observed for some general COX inhibitors, while the COX-II selective inhibitor NS-398 was essentially inactive (Table V). Although NDGA is a relatively poor inhibitor of cyclooxygenase catalytic ability, this result suggested that the dicatechols are inhibiting cytokine-stimulated COX-II expression.

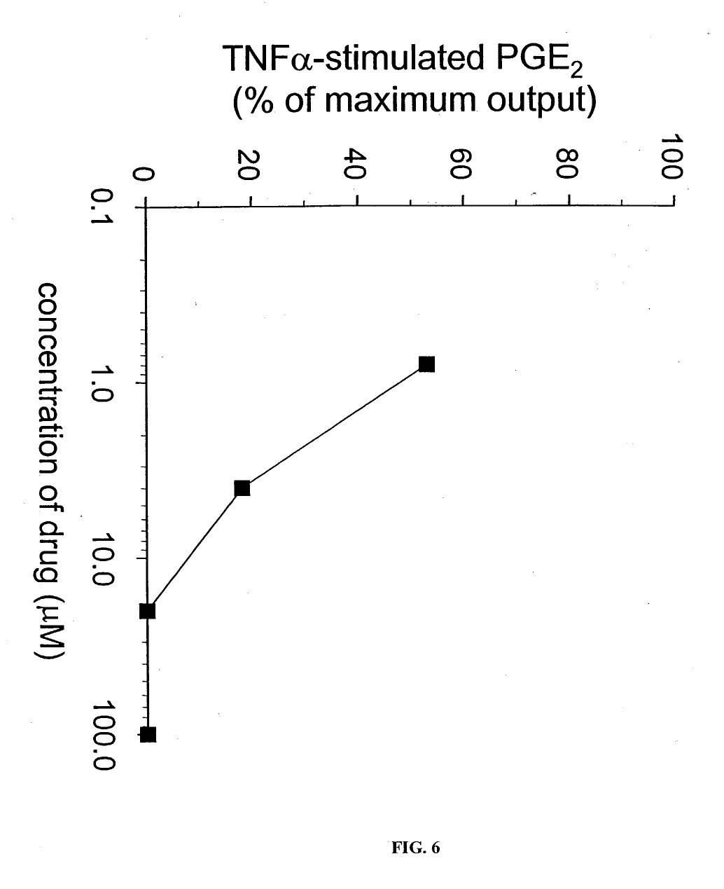

It was noted that NDGA is effective at submicromolar concentrations as an inhibitor of prostaglandin E2 (PGE2) release in TNFα-stimulated EOC-20 cells (FIG. 6). Prostaglandin E2, a product of the cyclooxygenation of arachidonic acid released from membrane phospholipids, plays major roles in regulating brain injury and inflammation. Although prostaglandin E2 has frequently been considered as a possible inducer of brain damage and degeneration, it may exert beneficial effects in the CNS. Indeed, in spite of its classic role as a pro-inflammatory molecule, several recent in vitro observations indicate that prostaglandin E2 can inhibit microglial activation.

Caffeic acid phenethyl ester (CAPE) was approximately as effective as curcumin, while other selective 5LOX inhibitors were bioactive but less potent. Interestingly, tetra-O-methyl NDGA (which does not inhibit lipoxygenase; Whitman et al. 2002) displayed modest bioactivity, though it was less potent than the parent compound (Table V). Variable activity was observed amongst the several archetypal nonsteroidal anti-inflammatory drugs that were tested. Indomethacin and ibuprofen displayed weak activity (Table V). A large series of classical free radical scavenging antioxidants (including monocatechols, SOD and catalase mimetics) displayed no activity against TNFα-stimulated microglia. Thus, the TNFα-antagonistic effect of NDGA is likely due to a combination of activities including but not restricted to the inhibition of 5LOX catalysis.

TABLE V
Efficacy of Various Antagonists of Arachidonic Acid Metabolism
Against TNFα-Stimulated Nitrite Productin by EOC-20 Microglia.

| Compound | Principal Target(s) | IC₅₀ (μM) | LD₅₀ (μM) |
|---|---|---|---|
| NDGA (natural) | 5LOX, RTKs | 3 | Nontoxic |
| NDGA (synthetic) | 5LOX, RTKs | 4 | Nontoxic |
| NDGA tetraacetyl ester | 5LOX, RTKs | 5 | Nontoxic |
| tetra-o-methyl-NDGA | — | 20 | 125* |
| Curcumin | 5LOX, RTKs | 14 | Nontoxic |
| curcumin diacetyl ester | 5LOX, RTKs | 14 | Nontoxic |
| caffeic acid phenethyl | 5LOX | 12 | Nontoxic |
| ester (CAPE) | | | |
| Zileuton | 5LOX | 105* | Nontoxic |
| MK-886 | FLAP | 41 | 40 |
| Sesamin | Fatty acyl Δ⁵ | 42 | Nontoxic |
| | desaturase | | |
| Aristolochic acid | PLA2 | 29 | 100 |
| Arachidonyl trifluoromethyl | PLA2 | 5 | 77 |
| ketone | | | |
| Indomethacin | COXI, II | 61 | 124* |
| Ibuprofen | COXI, II | Inactive | Nontoxic |
| NS-398 | COXII > COXI | 108* | 129* |
| Nimesulide | COXII > COXI | 20 | 178* |

*Values obtained by linear extrapolation from points ≦100 μM.

### Example VIII

The 5LOX Product Leukotriene B4 Stimulates TNFα-Mediated Nitrite Production in EOC-20 Cells

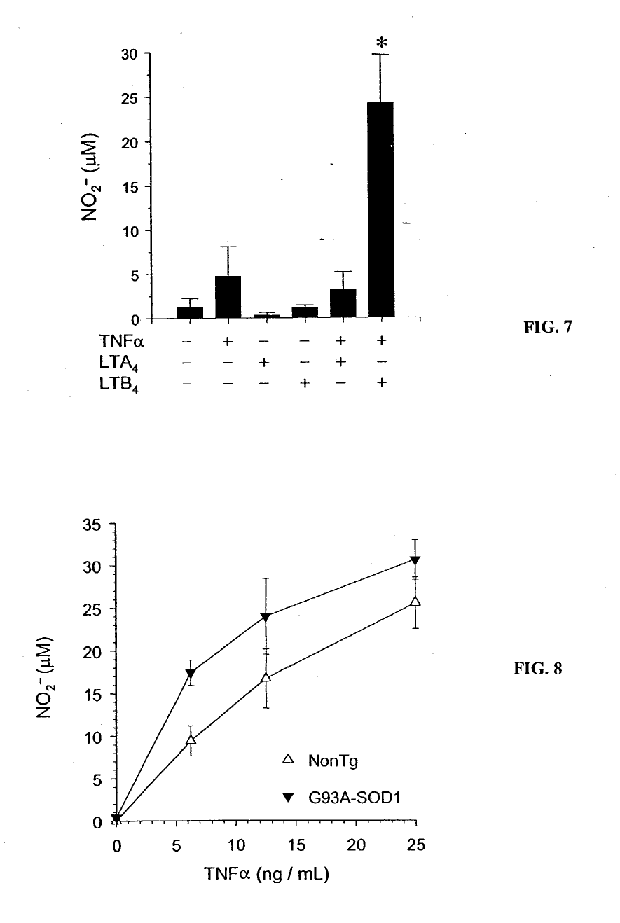

Because 5LOX inhibitors tended to antagonize TNFα, the converse experiment was performed of directly adding 5LOX metabolites to EOC-20 cells in the presence or absence of TNFα. LTB₄ was added to microglia at 10 μM and TNFα was added at a low dose (4 ng/mL) 10 min later. Nitrite was assayed at 24 h. Neither LTA₄ nor LTB₄ stimulated NO₂⁻ production autonomously; however, LTB₄ synergized with low-dose TNFα to produce a significantly greater nitrite yield than did the cytokine alone (FIG. 7). Similar examples exist in the literature of LTB₄ synergizing with IFNγ to activate macrophages (Talvani et al., 2002). Further studies may be conducted to confirm that 5LOX modulates TNFα using glia that do not express 5LOX (either isolated from 5LOX knockout mice or otherwise genetically manipulated).

The data indicates that 5LOX is intimately involved in the propagation of TNFα signals within microglia, and that appropriate 5LOX inhibitors might provide some protection against neuroinflammatory disease.

### Example IX

Primary Glial Cultures from G93A-SOD1 Mice are More Sensitive to TNFα Stimulation than are Glial Cultures from Nontransgenic Littermates

The inventors predicted that inflammatory signal transduction is perturbed in glia expressing mutant SOD1. This prediction has been tested using primary mixed glial cultures isolated from G93A-SOD1 mouse pups, or nontransgenic littermates. Such glial cultures are mostly astrocytes (at least 90%) with the remainder being mostly microglia (Robinson et al., 1999; Gabbita et al., 2002). More highly purified microglia can be cultured, but this requires larger numbers of mouse pups and more extensive culture manipulations.

To assess the effects of SOD1 mutations on TNFα signaling, mixed primary glia from G93A-SOD1 or nontransgenic mice were treated with increasing concentrations of TNFα. Nitrite was assayed in the culture medium after 30 h. As shown in FIG. 8, G93A-SOD1 glia produced significantly more NO₂⁻ in response to TNFα than did nontransgenic cells. At the lowest concentration of TNFα that was tested in this experiment, the G93A-SOD1 glia produced twice as much NO₂⁻ as did nontransgenic cells (FIG. 8). The relative difference in NO₂⁻ output between the two genotypes began to decrease at higher doses of TNFα, perhaps representing a saturation of the cell response at higher cytokine concentrations (FIG. 8). Future experiments may be conducted to more thoroughly explore the genotype-specific difference in glial response at lower concentrations of TNFα, where transgene-associated differences are likely to be more easily distinguished.

It is possible that the hypersensitivity of G93A-SOD1 cells stems merely from the overproduction of a transgenic protein, which would be irrelevant to the pathogenesis of ALS. Future studies, may more formally compare the cytokine sensitivity of G93A-SOD1 glia with that of cells expressing similar levels of wild-type human SOD1 transgenes. The data in FIG. 8 very clearly complement the inventors' findings of increased neuroinflammatory cytokine expression in G93A-SOD1 mouse spinal cord (described above). The data strongly suggest that SOD1 mutations somehow perturb signal transduction elements between the level of TNFα receptors and downstream nuclear transcription factors, with the result being a hypersensitivity to pro-inflammatory signal transduction.

### Example X

Proposed Model for the TNFα-LOX Signaling Axis in Microglia

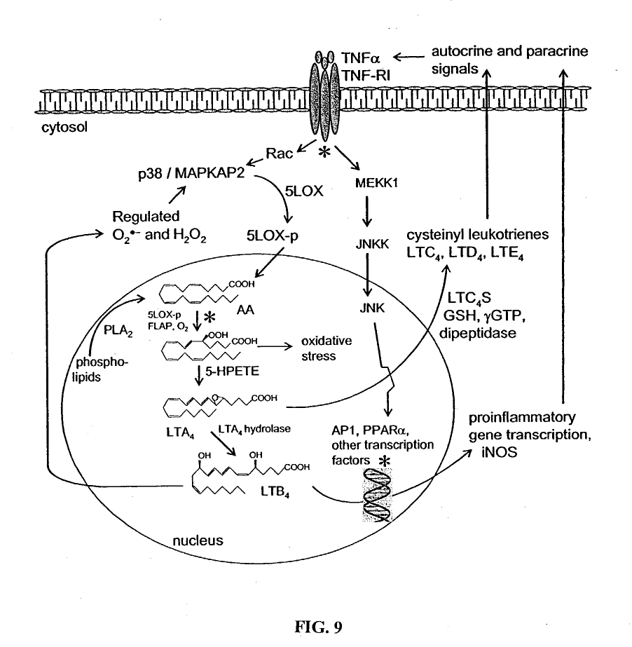

It is strongly suggested that lipoxygenase activity can modulate inflammatory signal transduction in macrophage or microglia cells. A model was then formulated to explain the data obtained in preliminary studies of the present invention, and to generate more testable hypotheses (FIG. 9). In this model, TNFα signals are transduced to the nucleus by parallel pathways: one branch activating 5LOX through p38-MAP kinase, and a separate branch activating other transcription factors including AP1 and PPARα (this latter portion of the model is largely based on findings of Funk and other investigators, e.g., Funk, (2001); Madamanchi et al., (1998); Rizzo and Carlo-Stella, (1996); Hallenbeck, (2002)). The diagram in FIG. 9 also incorporates the findings of Woo et al., (2000) who showed that LTB₄ is responsible for ROS generation in TNFα-stimulated fibroblasts, though the molecular target for LTB₄-sensitive ROS generation remains to be determined. The model explains why LTB₄ alone is insufficient to stimulate nitrite production, whereas the leukotriene is able to amplify a pre-existing stimulation along a TNFα-MAP kinase axis. This model also predicts that LOX inhibition alone will not completely abrogate a TNFα stimulus, but that dual inhibition of LOX and upstream receptor tyrosine kinases (RTKs) might well do so.

### Example XI

The Selective 5LOX Inhibitor NDGA Slows Disease Progression and Extends Survival in the G93A-SOD1 Mouse Model of ALS

The potency of NDGA in vitro inspired further study in vivo. Two separate studies were performed. In the first experiment, 10 G93A-SOD1 mice were randomized into groups receiving 10 mg/kg NDGA i.p. or vehicle alone, beginning at 90 D of age. NDGA was administered in 20% DMSO: 80% saline, as the compound has limited water solubility. Rotarod performance tests were conducted at 5 D intervals. The NDGA-treated animals exhibited a 38% reduction in the mean rate of motor functional decline at ages>100 D (FIG. 4). The median lifespan of the five NDGA-treated animals was 127 D, as compared to 121 D for the control group, representing a 20% extension of lifespan after the start of treatment.

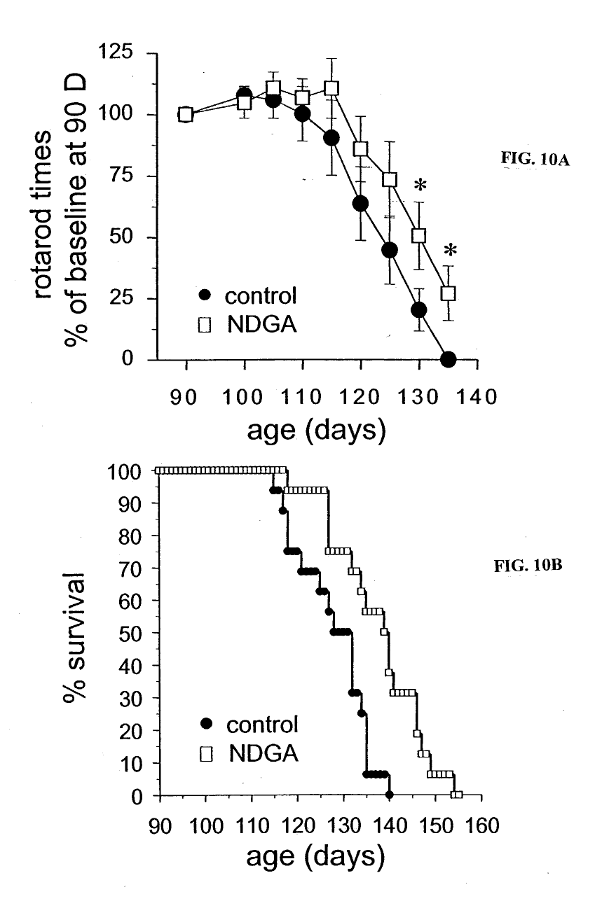

In the first study, the small number of animals did not allow rigorous statistical evaluation of the drug effect; animals seemed uncomfortable with repeated i.p. injections of the vehicle; and the study was not done in a rigorous observer-blinded fashion. Therefore, a second, larger study was conducted to overcome these limitations. NDGA was formulated into AIN93G laboratory mouse chow at 2500 ppm (approximately 40 mg/kg intake/mouse/day). This represents half the maximum concentration of curcumin that was found effective in an Alzheimer's mouse model (Lim et al., 2001). G93A-SOD1 mice and nontransgenic littermates were fed this NDGA-containing diet, or a control diet, beginning at 90 D of age. An observer blinded to the treatment groups tested the animals on the rotarod task at 100 D and every 5 days thereafter. As shown in FIG. 10A, oral NDGA significantly improved rotarod performance in an age-dependent fashion (p<0.03 for the drug effect by repeated measures of analysis of variance; p<0.001 for a drug X age interaction). Survivability was likewise extended by NDGA, FIG. 10B, (median age of death for control animals=127 days; for NDGA-treated animals=140 D; RR=0.27; p<0.01 by logrank analysis). No weight changes were observed in nontransgenic or transgenic mice as a function of NDGA in the diet; and no pathological effects of the drug were observed at necropsy. This 13 D extension of lifespan is the same magnitude of benefit observed with oral administration of riluzole beginning at 50 D (Gurney et al., 1996). It represents a 32% prolongation of lifespan after start of NDGA treatment at 90 D. In further comparison, the NDGA benefit is similar to that reported for minocycline, which increases lifespan of the fast-progressing G93A-SOD1 mouse by 11 days when administration is begun at seven weeks of age (Zhu et al. 2002). Animal weights were not significantly affected by oral NDGA administration While G93A-SOD1 mice display measurable muscle weakness between 90-110 D of age, obvious signs of paralysis usually become evident near 115 D. This event can be defined by a number of indicators including an altered leg-splaying response when the mouse is lifted by the tail (Gurney et al., 1996). NDGA significantly delayed the onset of frank paralysis, as indicated by leg-splaying criteria (Table VI). Moreover, the paralytic phase of disease (time between onset of paralysis and death) was extended approximately 40% by oral intake of NDGA, and this effect was marginally significant (p<0.06; Table VI). This study differs from most experiments performed on the G93A-SOD1 mouse in that NDGA treatment was begun at 90 D of age, a time when the transgenic mice are measurably impaired relative to their nontransgenic littermates. Notably, no other systemically administered drug has ever shown efficacy in the ALS mouse, when administration was begun at a late date. This is a very important point, as most forms of human ALS are sporadic and prophylactic treatment is not practical. In the NDGA study, the drug extended median lifespan by 13 D. This is virtually the same extension observed when riluzole was administered to G93A-SOD1 animals prophylactically beginning at 50 D of age (Gurney et al., 1996).

TABLE VI
Oral NDGA Affects the Onset of Frank Paraylsis as Well as
Duration of the Paralytic Stage of Disease in G93A-SOD1 Mice.

| | Control | | NDGA | |
|---|---|---|---|---|
| Onset of frank paralysis | | | | |
| mean ± SD (p) | 115.9 ± 7.4 | D | 121 ± 7.1 D | (0.029) |
| median | 112 | D | 120 D | |
| Time between onset and death | | | | |
| mean ± SD (p) | 11.5 ± 5.7 | D | 16.5 ± 7.9 D | (0.053) |
| median | 10 | D | 14 D | |

In a separate experiment, NDGA was administered intraperitoneally to a small group of G93A-SOD1 mice (5 control and 5 drug-treated animals; 10 mg/kg 5 days/week beginning at 90 D). In this paradigm, NDGA also extended survival although logrank statistics were not formally significant (median lifespan after start of treatment=31 D in control animals and 37 D in NDGA-treated animals; p=0.074 by logrank test). Rotarod performance was not significantly affected by i.p. administration of drug alone, though there was a significant (p<0.01) drug x time interaction term when the data were analyzed by repeated measures analysis of variance. The i.p. administration of NDGA was somewhat affected by the inclusion of DMSO in vehicle; G93A-SOD1 mice receiving DMSO alone died approximately 10 D sooner than normally would be expected from these animals. Taken together, the data strongly indicate a positive effect of NDGA on the prognosis of disease in rapidly-progressing G93A-SOD1 mice.

### Example XII

NDGA Suppresses Astrogliosis and Microglial Proliferation in G93A-SOD1 Mice

Astrogliosis, characterized in part with the enhanced expression of glial fibrillary acidic protein (GFAP), is a homotypic response of astroglia to diverse types of central nervous system injury (Little and O'Callagha, 2001). Astrogliosis is a major tissue-level phenotype associated with G93A-SOD1 transgene expression (Hall et al., 1998; Drachman et al., 2002). Recent studies of cyclooxygenase II inhibitors have shown that NSAID suppression of astrogliosis correlates with improved prognosis in the G93A-SOD1 mouse model (Drachman et al., 2002). Accordingly, astrogliosis was investigated immunochemically as a function of NDGA administration.

Mice were anesthetized with pentobarbital and perfused transcardially with 4% paraformaldehyde in phosphate buffer. The lumbar region (L1-L5) was processed for paraffin embedding. Immunochemistry was performed on 8 mm-thick sections, using commercially available antibodies, and tissue sections were routinely counterstained with hematoxylin and eosin. Polyclonal anti-SOD1 IgG was purchased from Chemicon (Temecula, Calif.). Polyclonal anti-5LOX IgG was purchased from Cayman Chemical (San Diego, Calif.). Monoclonal anti-5LOX was obtained from Transduction Laboratories (Lexington, Ky.). Polyclonal antibody against glial fibrillary acidic protein (GFAP) was purchased from Research Diagnostics International (Flanders, N.J.). FITC-conjugated anti-F4/80, which recognizes a microglial cell surface antigen (Drachman et al., 2002), was purchased from Serotec (Raleigh, N.C.). Positive control for 5LOX Western blots was SL-29 fibroblast lysate (Transduction Laboratories, provided with the antibody). Electrophoresis was performed on 4-20% gradient polyacrylamide gels, and bands were visualized with chemiluminescence detection reagents (Amersham).

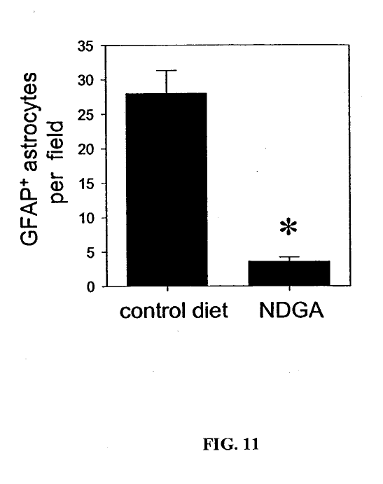

As shown in FIG. 11, oral NDGA significantly diminished astrogliosis in the lumbar spinal region of 120 D old G93A-SOD1 mice, relative to transgenic mice that received the control diet.

Microglial proliferation is another neuroinflammatory feature inherent to motoneuron disease in the G93A-SOD1 mouse (Drachman et al., 2002). Microglia cells in the G93A-SOD1 mouse spinal cord were immunochemically labeled using fluorophore-conjugated antibody against the macrophage and microglia surface antigen F4/80 (Drachman et al., 2002). Oral administration of NDGA diminished the number of F4/80-positive microglia in lumbar sections of G93A-SOD1 mouse spinal cord, when tissue was assessed at 120 D of age. Thus, presumptively beneficial effects of NDGA can be observed at the tissue level as well as at the behavioral level in G93A-SOD1 mice.

### Example XIII

5LOX Protein and Message is Increased in G93A-SOD1 Mouse Spinal Cord

NDGA was administered to G93A-SOD1 mice based on its ability to antagonize TNFα in a microglia cell culture system, without any a priori considerations regarding the molecular targets of action. As discussed above it is likely that the TNFα-antagonizing effects of NDGA do not map exclusively to 5LOX; nonetheless 5LOX is the primary acknowledged target for NDGA. The inventors therefore decided to investigate whether 5LOX expression is affected by the G93A-SOD1 transgene. Western blot analyses indicated a 5-fold elevation of 5LOX protein in G93A-SOD1 mouse spinal cord at 80 D and a 2-fold elevation at 120 D. The decrease at 120 D relative to 80 D may reflect loss of 5LOX expressing neurons. Essentially no reactivity was observed when the same samples were probed with antibodies against 12-LOX and 15LOX.

As further confirmation that 5LOX expression increases in G93A-SOD1 mice, semiquantitative reverse transcriptase polymerase chain reaction (RT-PCR) analysis was undertaken using 5LOX specific primer pairs. Total RNA was isolated from the upper spinal cords of non-transgenic control and G93A±transgenic mice by using TRI Reagent (Sigma, St Louis, Mo.) according to the supplied protocol. 5 μg samples of RNA were reverse-transcribed using oligo(dT)₁₅ to prime the reaction in the presence of AMV-reverse transcriptase (Roche, Indianapolis, Ind.) following the manufacturer's protocol. On completion, each reaction was diluted to a final volume of 50 μl with TE buffer (10 mM tris, 1 mM EDTA, pH 8.0). PCR amplification of a 309 bp 5LOX gene product from the above-described mouse cDNAs was accomplished with Taq DNA polymerase (Roche, Indianapolis, Ind.) 2.5 units/reaction, utilizing the supplied buffer and final concentrations of 1.5 mM MgCl₂, 0.2 mM each dNTP, 0.3 μM each primer. Final reaction volumes were 50 μl. Mouse 5LOX primers were GGCACCGACGACTACATCTAC (forward) (SEQ ID NO: 1) and CAATTTTGCACGTCCATCCC (reverse) (SEQ ID NO: 2). β-actin primers (CGGCCAGGTCATCACTATTG—forward (SEQ ID NO: 3), ACTCCTGCTTGCTGATCCAC—reverse (SEQ ID NO: 4)) yielding a 353 bp PCR product were used as normalization controls. The number of PCR amplification cycles was empirically determined to yield detectable product bands that were approximately linear with respect to initial cDNA concentration. For 5LOX, optimal cycling conditions were: 2 min. at 94° C., 1 cycle; 1 min. at 94° C., 1 min. at 56° C. and 1 min. at 72° C. for 27 cycles; 7 min. at 72° C., 1 cycle. Conditions for actin primers were the same except that an annealing temperature of 54° C. was used and 24 cycles were performed. Samples of 25 μl from each reaction were electrophoresed in 2% agarose/TBE gels for 1.5 hrs., stained with ethidium bromide and photographed with a NucleoVision (Nucleotech, Westport, Conn.) imaging system.

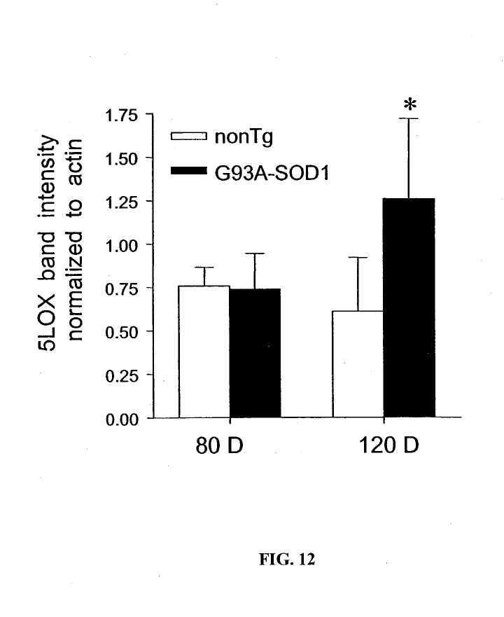

As FIG. 12 illustrates, 5LOX mRNA is increased at least 2-fold at 120 D relative to the levels in nontransgenic mouse spinal cord. Taken together, the data suggest that antagonism of 5LOX likely explains some of the beneficial effects of NDGA in this mouse model.

### Example XIV

SOD1 Binds 5LOX in vitro and in vivo: A Mechanism for Disruption of the TNFα-5LOX Axis by SOD1 Mutations

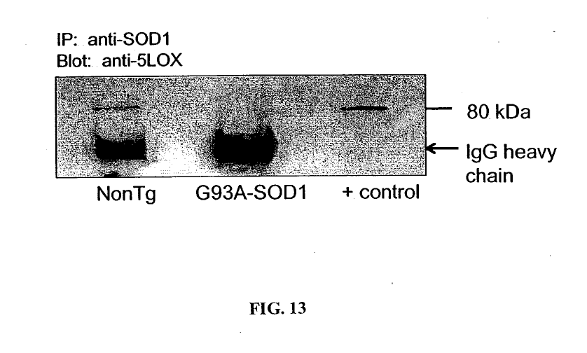

One of the most important, unanswered questions in ALS research is: Why do SOD1 mutations in FALS so accurately reproduce the phenotype of sporadic ALS? Despite great effort, no defects (genetic or post-translational) have been found in SOD1 of sporadic ALS patients. Thus, it is likely that mutant SOD1 perturbs a pathway(s) that is coincidentally dysfunctional in SALS. The data suggests that the TNFα-5LOX axis may be a pathway whose function in FALS and SALS might be worthy of consideration. It is conceivable that altered protein-protein interactions elicited by aggregating mutant SOD1 could affect this, or similar inflammatory components, thus reproducing a defect that occurs for some other reason in SALS. To probe putative SOD1-lipoxygenase interactions, spinal cord lysates from 120 D old G93A-SOD1 mice, or nontransgenic littermates, were immunoprecipitated with polyclonal anti-SOD1 IgG (Chemicon). The immunoprecipitates were probed with a monoclonal antibody against 5LOX (Transduction Labs). Lysates from nontransgenic animals contained a protein that was immunoreactive with anti-5LOX and which corrugated exactly with a 5LOX standard (FIG. 13). Lysates from G93A-SOD1 mice contained very little of this protein (FIG. 13). The relative lack of immuno-recognizable 5LOX in the transgenic mouse spinal cord lysate may represent an artifact of competition between the high levels of free SOD1 in the transgenic cord, and LOX-bound SOD1, for the immunoprecipitating antibody. Nonetheless, the data in FIG. 13 provide the first evidence for an interaction between SOD1 and 5LOX in any cell or tissue system. Thus far, the converse immunoprecipitation of SOD1 using anti-5LOX has been unsuccessful due to the inefficiency of commercial 5LOX antibodies when applied to spinal cord lysates.

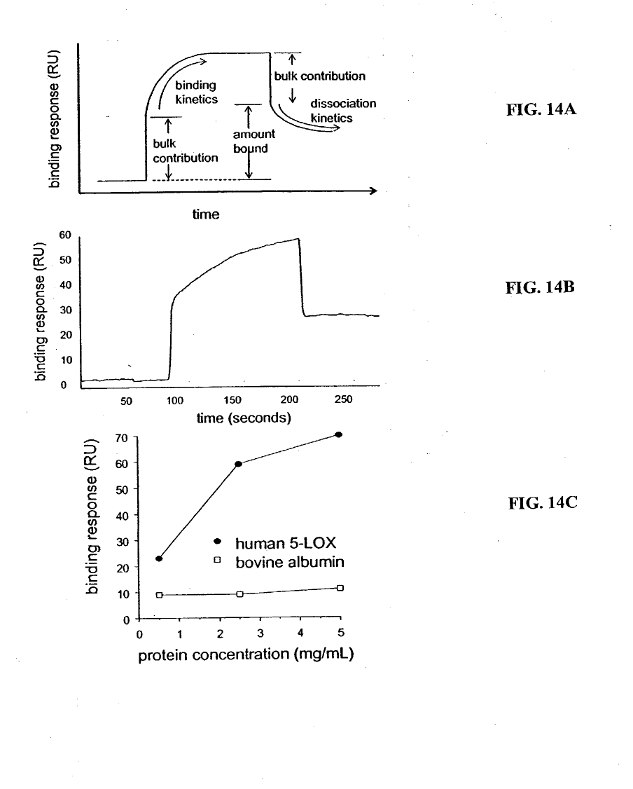

Using several techniques that allow the study of protein-protein binding interactions in vitro, the interaction of SOD1 with 5LOX may be investigated. One approach involves the use of BIAcore instrumentation (Amersham-Pharmacia, Upsala, Sweden). The BIA (biomolecular interaction analysis) system consists of a solid-phase support upon which a protein (the ligand) can be covalently immobilized. This surface is placed in contact with a microfluidic cartridge, which dispenses a second protein (the analyte) across the binding surface. Adherence of the analyte to the immobilized ligand is measured by surface plasmon resonance (SPR) spectrometry. The surface plasmon resonance detector responds to refractive index changes in the vicinity of the sensor surface as the immobilized species interacts with its binding partner in the fluid phase. Data output from the BIAcore instrument takes the form of “sensorgrams” (FIG. 14A). Sensorgrams can be interpreted qualitatively, to assess whether a nonspecific binding event occurs, or quantitatively, in order to measure binding affinity.

In a previous experiment, human wild-type SOD1 (purified to homogeneity from human erythrocytes) was covalently immobilized to the BIAcore chip via surface lysine residues, using standard carbodiimide coupling chemistry. Human recombinant 5LOX (Cayman Chemical) was passed over the SOD1 surface. In control experiments, bovine serum albumin (BSA) was passed over the surface. SPR analysis indicated a strong nonspecific binding of 5LOX to immobilized SOD1 (FIGS. 14B-14C). Experiments to quantitate the binding affinity are conducted. Preliminary Scatchard analysis suggests an upper bound on the K_{d} is 2×10⁻⁵ M but the actual affinity is likely to be stronger, since ligand immobilization often tends to sterically hinder the approach of analyte to a binding surface. It can be seen from typical sensorgrams that 5LOX binding is very strong, as indicated by the very slow dissociation kinetics of 5LOX bound to the SOD1 surface (FIG. 14C). In fact, it was found, that complete dissociation of bound 5LOX is not possible under standard regeneration conditions, which employ 3M KSCN as a dissociation-promoting reagent.

In the converse experiment, 5LOX was immobilized to the BIAcore chip and SOD1 was used as the fluid-phase analyte. In this configuration, very poor binding was observed. These data indicate that surface lysines of 5LOX are involved with binding of SOD1, such that attachment of these residues to the BIAcore surface would block SOD1 access. Such phenomena often occur in BIAcore experiments and represent one limitation of the technique.

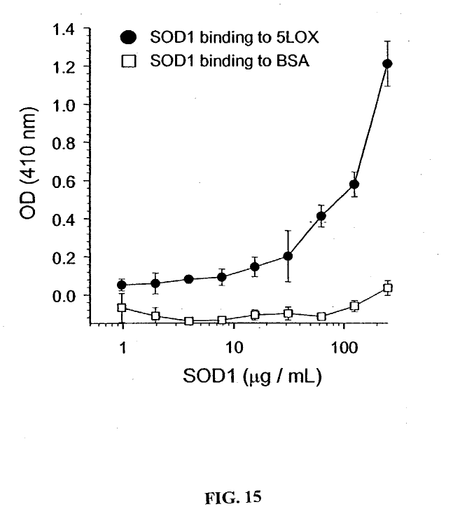

In order to circumvent these limitations of the BIAcore technique, a conceptually different strategy was employed to assess SOD1 binding to immobilized 5LOX. Standard 96 well polypropylene microtiter plates were coated with 5LOX by overnight incubation with the protein, which allows adsorption of the 5LOX through mostly hydrophobic interactions with the plate surface. As a control, half of each microplate was coated with BSA. The entire plate was then blocked with BSA, and subsequently incubated for 1 H with various concentrations of SOD1 dissolved in physiologic saline. Binding of SOD1 to the 5LOX-coated (or BSA-coated) microplate wells was quantified using alkaline phosphatase-conjugated anti-SOD1 polyclonal IgG, and p-nitrophenol as the chromophore. FIG. 15 illustrates preliminary binding data obtained from this experiment. SOD1 bound strongly to 5LOX-coated microplates but not to BSA-coated surfaces. Further experiments may be conducted to approximate a 5LOX-SOD1 binding affinity; previous Scatchard analysis yield an estimate for K_{d}=1.4×10⁻⁶ M (i.e., 10 times stronger than the BIAcore estimate). Experiments may also be conducted to assess differences in 5LOX binding to wild-type versus mutant SOD1.

### Example XV

5LOX Dysregulated in Alzheimer's Disease: Data from APP/PS1 Mice and Humans

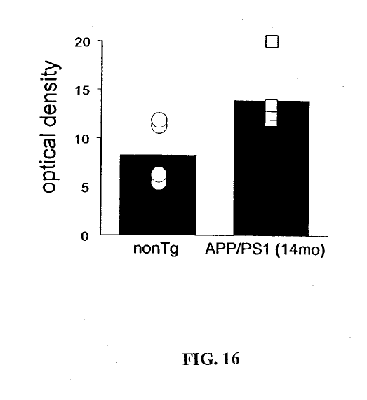

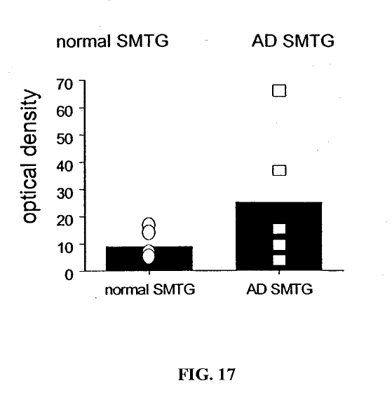

The inventors investigated whether 5LOX is dysregulated in mouse models for Alzheimer's disease. Cortical tissue from amyloid precursor protein/presenilin 1 (APP/PS1) double transgenic mice was probed by Western blot for 5LOX protein. These mice develop amyloidopathy and cognitive deficits at 12-16 months of age (Morgan et al., 2000). At 14-15 months of age, cortical 5LOX was elevated by 80% although this was not formally significant (FIG. 16). The inventors have also investigated 5LOX dysregulation in human AD brain tissue obtained under rapid postmortem protocols. Western blots indicate highly variable expression of 5LOX in AD brain cortex, though the average level of 5LOX was 2.8-fold greater in AD than in normal cortex (FIG. 17). These data suggest that perturbations in 5LOX may be common to multiple, age-related neurodegenerative conditions including Alzheimer's disease.

### Example XVI

NDGA Blocks Aβ-Induced Neurotoxicity

Alzheimer's disease is caused, in part, by accumulation of β-amyloid peptides (A-beta or Aβ). Frautschy et al. (2001) have described a rat model of amyloid-induced neurotoxicity which is useful for the purpose of evaluating potential Alzheimer's therapeutics. In this model, rats are infused intracerebroventricularly (ICV) with Aβ adsorbed to an apo-lipoprotein carrier. Thus, to determine whether dicatechol (e.g., microglial inhibitor nordihydroguaiaretic acid) blocks Aβ-induced neurotoxicity the Frautschy model was used. Six to eight months old adult Sprague-Dawley male rats (Harlan, Ind.), weighing 250-275 grams, were divided into four groups. Each group consisted of six rats fed either a control or an NDGA supplemented diet (Table VII).

TABLE VII
Design of a study to test dicatechol efficacy against
Alzheimer's disease-associated amyloid neurotoxicity.

| Treatment | Treatment intracerebroventricular, | |
|---|---|---|
| Group | 0.25 μl/h, 28 days) | Diet |
| Group-1 | Vehicle [0.35% BSA and HDL (0.1 μg/h)] | Control |
| Group-2 | Aβ 40 (25 ng/h & Aβ 42 (37.5 ng/h) | Control |
| Group-3 | Aβ 40 (25 ng/h) & Aβ 42 (37.5 ng/h) + human | Control |
| | ApoE4 (6 ng/h) | |
| Group-4 | Aβ 40 (25 ng/h) & Aβ 42 (37.5 ng/h) + human | 0.25% |
| | ApoE4 (6 ng/h) | NDGA |

NDGA was formulated into laboratory rat chow at 2500 ppm (0.25% of diet, approximately 40 mg/kg intake/rat/day). Rats were trained on a radial eight-arm maze as described previously. After they achieved 0-2 error level, they underwent surgery for implantation of the cannulae and osmotic pumps. Twenty-four hours after surgery, they were started on the dietary treatment.

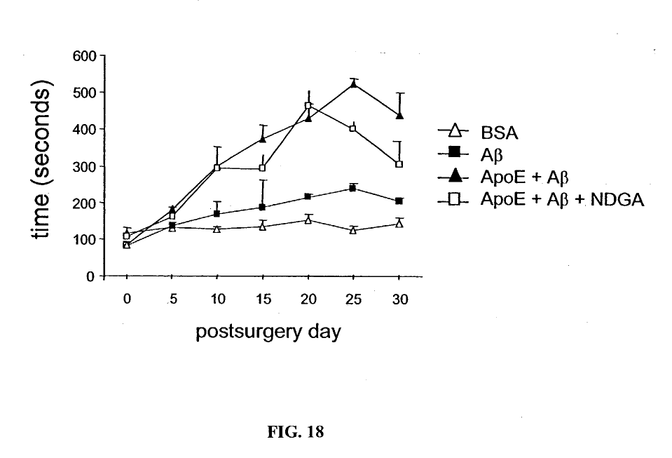

Rats treated with Aβ40 and Aβ42 and ApoE4 showed significantly impaired performance. There was a decrease in the number of errors following termination of the treatment on day 28. NDGA suppressed reference errors on the day 25 and the day 30 of the treatment, though the differences were not significant due to a small number of animals. NDGA also produced a significant decrease in the number of working memory errors and latency on day 25 (FIG. 18).

Time and error scores from six trials for each experimental group were subjected to analysis of variance (ANOVA). Tucky-Kramer test for multiple comparisons was used as a posthoc test. Performance in the memory task was analyzed separately over all six trials.

This study provides evidence that an increase in ApoE levels in ApoE4 carriers increases neurotoxic effects of Aβ. This study also supports the involvement of neuroinflammation in ApoE-4 induced increased neurotoxicity of Aβ 40+42 peptides. Several studies have reported the evidence of microglial activation, and involvement of IL1, IL6 and TNFα in the pathogenesis of Alzheimer's disease. The data strongly suggest that 5-lipoxygenase is intimately involved in the neurotoxic response to Aβ (40+42)+ApoE-4 infusion. Thus, it is proposed the appropriate anti-inflammatory agents (e.g., 5LOX inhibitors) might offer a therapeutic benefit to AD patients.

### Example XVII

NDGA Protects Against Huntington's Disease and Parkinson's Disease-Associated Striatal Damage

Huntington's disease and Parkinson's disease involve damage to the substantia nigra and nigrostriatal portions of the brain. Damage to these areas can be selectively produced in rats and mice by intraperitoneal (I.P.) injection of 3-nitropropionic acid (3NP; Beal et al., 1993). This provides a means of testing potential drugs for neuroprotective benefit against Huntington's disease and Parkinson's disease. The inventors therefore determined whether microglial inhibitor nordiydroguariacetic acid protects against an animal model of Huntington's disease and Parkinson's disease-associated striatal damage.

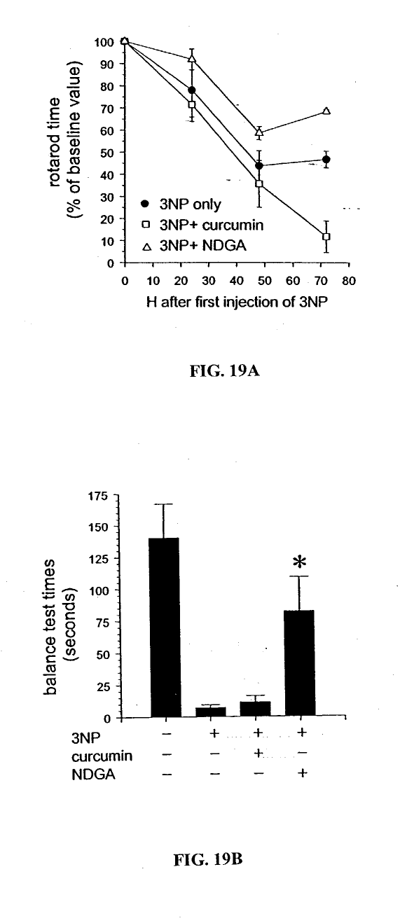

C57/B6 mice were fed a defined AIN-93G diet for one month, or the same diet containing 2500 ppm curcumin or NDGA. Mice were then injected with 50 mg/kg 3NP intraperitoneally, every 12 h for 5 days. Motor performance was assessed daily by a rotarod task and balance was assessed by a balance wire task. In the rotarod task (Hensley et al. 2002), animals were placed on a bar rotating at 1 rpm and accelerating 10 rpm/minute until the animal fell from the bar. In the balance beam test, mice were placed on a 2-cm balance beam by suspending them by the tail, orienting them perpendicular to the beam, and placing the forepaws followed by hindpaws on the balance beam. Mice were left on the beam until they fell onto a soft cushion 75 cm below or until 5 min elapsed. The longest time of each pair of trials was used for statistical analysis. As shown in FIG. 19, oral NDGA significantly improved functional ability in both tasks while curcumin had no effect on the balance test and possibly a detrimental effect on the rotarod test.

### Example XVIII

Improve Prognosis in the G93A-SOD1 Mouse Model of ALS by Genetic Ablation of Either TNFα or 5LOX I

Well-characterized transgenic mice exist that have targeted disruptions in either the TNFα gene or the 5LOX gene. Two such mouse lines can be bred into the G93A-SOD1 mouse, and progeny phenotyped with respect to survivorship, motor performance, and onset of paralytic disease (Table VIII). Neuroinflammatory indicators can be assessed using multiprobe ribonuclease protection assays (RPAs) and multiplex cytokine arrays, which have been previously optimized for neurochemical assessment of disease in the G93A-SOD1 mouse. Any delay of disease onset or progression that results from disruption of either gene constitutes validation of a new molecular target that could be exploited for pharmacological benefit in treatment of ALS.

TABLE VIII
Summary of the Genotypes of 5LOX and TNFα Knockout Mice

| | G93A-SOD1 | 5LOX | TNFα |
|---|---|---|---|
| A | NonTg | +/+ | +/+ |
| B | +/− | +/+ | +/+ |
| C | +/− | −/− | +/+ |
| D | +/− | +/+ | −/− |

Animals. Table VIII summarizes the genotypes of mice that can be generated. Founder mice containing targeted disruption of either the 5LOX or TNFα gene can be purchased as breeder pairs from Jackson Laboratories (Bar Harbor, Me.). The precise strains may comprise the B6;129S2-ALOX5^{tm1Fun} for the 5LOX knockout animal (Chen et al., 1994) and the B6;129S6-TNF^{tm1Gk1} strain for the TNFα knockout animal (Pasparakis et al., 1996). The two knockout animals may be obtained in the homozygous condition. Additionally, B6129SF2/J mice can be obtained for use as a control strain.

Mice homozygous for the TNF^{tm1Gk1} targeted mutation are viable and fertile, and show no blatant phenotypic abnormalities in the absence of an applied inflammatory or carcinogenic stress. Additionally, obese TNFα-knockout mice display reduced insulin levels and deficient glucose clearance responses relative to obese wild-type mice. Similarly, 5LOX deficient homozygous mice are viable and fertile, and show no blatant phenotype but are selectively resistant to arachidonic acid-induced inflammation (though lethality in response to endotoxic shock is not different from that of wild-type mice).

Homozygous 5LOX or TNFα disrupted mice can be bred to G93A-SOD1 mice (obtained from Jackson Laboratories, Bar Harbor, Me. and maintained as hemizygotes in the C57B6/SJL strain). The F1 progeny can be genotyped with respect to mutant SOD1 transgenes, and 5LOX or TNFα as appropriate. Survival and onset-of-paralysis parameters can be monitored closely in the G93A-SOD1 positive F1 mice. The littermates that are heterozygous with respect to either 5LOX or TNFα, and which are positive for the mutant SOD1 transgene, can be back-crossed to homozygous TNFα or 5LOX mice. The resulting members of the F2 generation which are positive for G93A-SOD1 and homozygous deficient in TNFα or 5LOX may be fully assessed for motor functional ability, survivorship and onset-of-paralysis criteria.

A third breeding protocol may cross G93A-SOD1 mice with B6129SF2/J mice, and backcross the G93A-SOD1 positive members of the F1 generation with the B6129SF2/J parent strain. The F2 offspring that are G93A-SOD1 positive and wild-type with respect to both TNFα and 5LOX may be used as controls for the corresponding knockout animals.

All animals of appropriate genotype may be trained to the rotarod task at 40 D of age, and assessed at weekly intervals until dead or no longer able to perform the task (as described previously). Rotarod testing can be performed using a commercially available device (Columbus Instruments). The animals may be placed on a horizontal rod set to spin about its long axis, initially at 1 rpm. The revolution rate may be increased at a constant rate of 1 rpm every 10 sec and the experiment continued until the animal falls off the rod. Each animal may be assessed in triplicate trials at each test period. Animal weights may also be recorded at each test period. Onset-of-paralysis may be defined by altered leg-splaying response (Gurney et al., 1996) as judged by an observer blinded to genotype groupings. Animals may be killed when they can no longer perform the rotarod task or right themselves within 10 sec of being placed on their sides (Gurney et al., 1996).

Statistics. A minimum of 20 F2 animals may be utilized in each breeding scheme. Survivorship parameters may be statistically assessed using classical logrank methods (Mantel-Haenszel and Kaplan-Meiers statistics). Rotarod performance measures may be compared using repeated measures analysis of variance. Onset-of-paralysis data, and the duration of paralytic phase (onset to death), may be statistically compared using standard t-tests and Mann-Whitney tests in situations wherein the data does not follow a normal Gaussian distribution. All statistics routines can be performed using GraphPad Prism™ Statistical Applications Software (GraphPad Inc., San Diego, Calif.).

Ribonuclease protection assays for apoptosis and neuroinflammation. As described previously, the G93A-SOD1 mouse experiences hallmarks of neuroinflammation in the timeframe of neurodegeneration. The inventors have had considerable success in monitoring this type of process at the mRNA level using multiprobe ribonuclease protection assays (RPAs). The RPA approach allows the quantification of panels of up to 12 cytokine or apoptosis-associated mRNA species simultaneously. With respect to cytokines, the following probes may be included: IL1α, IL1β, IL1RA, IL2, IL4, IL10, IL18, MIF, IFNγ, TNF-RI, TNFα. With respect to indexing apoptosis in mouse spinal cord, RPAs may be used to determine expression of caspase and other proteins associated with apoptosis (caspase 1, 2, 3, 6, 7,8, 11 and 12; Fas, fas-ligand, bcl, bax). Spinal cord tissue (the lower ½ spinal cord) may be lysed in TRIzol™ mRNA isolation reagent (Life Technologies, Gaithersburg, Md.) with a Dounce-type homogenizer. Total RNA in the resulting extract may be quantified spectrophotometrically at 260 nm. A panel of apoptosis-associated RNA species can be detected using a commercially-available multiprobe ribonuclease protection assay system (Riboquant™, Pharmingen, San Diego, Calif.). Radiolabeled probes can be synthesized from DNA templates containing a T7 RNA polymerase promoter (Pharmingen, San Diego, Calif.). Templates can be transcribed in the presence of 100 μCi [γ-³²P]UTP to yield radioactive probes of defined size for each mRNA. Probes can be hybridized with 5-10 μg total RNA, then treated with RNAse A and T1 to digest single-stranded RNA. Intact double-stranded RNA hybrids can be resolved on 5% polyacrylamide/8 M urea gels. Dried gels can be developed using a phosphorimager (Molecular Dynamics, Sunnyvale, Calif.) and bands quantified using instrument-resident densitometry software (ImageQuant™, Molecular Dynamics). Within each sample, the density of each apoptosis-associated mRNA band is be divided by the sum of the L32+GAPDH bands. The same tissue may be separately probed for presence of eight pro- and anti-inflammatory cytokines (interleukins 1α, 1β, IL6, IL8, IL10, IL12, IFNγ, IL1 receptor antagonist). Each RPA may compare five animals from each group: G93A-SOD1 animals on basal vs. supplemented diet; and nontransgenic animals on basal vs. supplemented diets.

Cytokine protein expression arrays. Spinal cord lysates can be analyzed for 17 cytokines and chemokines in a simultaneous, multi-plexed format utilizing a novel microbead and flow based protein detection system (Bio-Plex™ System, Bio-Rad Laboratories Inc.). In this quantitative assay system, microcarrier beads are encoded with a set of three fluorophores, with distinguishable yellow-green fluorescence maxima. Because the proportions of the three labels can be precisely controlled, a series of 17 distinct microbead populations can be created that are separable by instrumental methods. Each of the 17 microbeads is conjugated to a specific antibody directed against a cytokine or chemokine target. Aliquots of sample are incubated with a microbead mixture; the beads are then separated by centrifugation, washed, and labeled with phycoerythrin-conjugated secondary antibody. The Bio-Plex system instrumentation incorporates fluidics, laser excitation, fluorescence detection, and digital signal processing in a manner that allows for the individual scanning and identification of individual microbeads. Each bead is individually identified based on its internal fluorescence signature, and the phycoerythrin reporter signal associated with that bead is quantitated. A minimum of 100 microbeads per each of the 17 targets is analyzed in each sample. All samples of spinal cord lysate may be assayed in triplicate. Observed concentrations of each target cytokine and chemokine can be determined based on an appropriate set of recombinant mouse cytokine and chemokine internal standard curves.

Histochemical assessment of gliosis. Animals may be killed by pentobarbital injection, perfused with saline, and the spinal cords removed and processed for histochemical assessment of gliosis using fluorescent-labeled monoclonal antibodies against glial fibrillary acidic protein (GFAP). This procedure has been used to index gliosis in the G93A-SOD1 mouse (Drachman et al., 2002) and is responsive to oral administration of the COX II inhibitor celecoxib (Drachman et al., 2002). Thus, GFAP is a validated marker for assessing the response of G93A-SOD1 mouse nervous system to experimental anti-inflammatory drugs. Serial sections can be stained for GFAP and the number of GFAP-positive cells per section scored by an observer blinded to the sample identity.

Assays for protein oxidation. Tissue homogenates from spinal cord, brain, and peripheral tissues can be assayed for protein carbonyl content using a biotin hydrazide method (40). Homogenates are made in 10 mM sodium acetate buffer pH 7.2 containing 0.1% triton X-100 and mammalian protease inhibitor cocktail (Sigma). Samples are adjusted to 2 mg/mL protein after Lowry assay and mixed 1:1 with 20 mM MES pH 5.5. To these samples are added a 1/10 volume of 50 mM biotin hydrazide (Molecular Probes, Eugene, Oreg.) dissolved in DMSO. Samples are incubated with gentle agitation overnight (16 H) at 37° C. Samples are then electrophoresed on 4-20% gradient polyacrylamide gels and blotted with streptavidin-conjugated horseradish peroxidase (BioRad).

Assays for leukotriene products. Leukotrienes A4, B4, C4, D4 and E4 can be assayed in spinal cord lysates using commercially available ELISA systems (Cayman Chemical, San Diego, Calif.). Whole cord lysates are prepared immediately before the assay. Standard lysis buffer contains 10 mM sodium acetate pH 7.4, 0.1% triton X-100, and 0.5 mM butylated hydroxytoluene (BHT) as an antioxidant to prevent artifactual oxidation of arachidonic acid. Lysates from 120 D old G93A-SOD1 animals, that are wild-type with respect to both TNFα and 5LOX, are compared to lysates from G93A-SOD1 animals from which TNFα or 5LOX has been genetically ablated.

Assessment of SOD1 copy number in 5LOX and TNFα-deficient animals. It is conceivable, but not likely, that the breeding scheme outlined above may result in decreased expression of the mutant G93A-SOD 1 transgene in 5LOX and TNFα-deficient animals. This would represent a trivial explanation for any protective benefit of the genetic ablations. To check for such an artifact, spinal cord lysates from 120D old animals of each genotype can be probed by Western blot methods using monoclonal IgG specific to human SOD1 (Sigma Chemical). Additionally, mRNA transcripts for human SOD1 can be monitored by Northern blot methods.

### Example XIX

Three Distinct 5LOX Inhibitors (NDGA, Curcumin, and Zileuton) Improve Prognosis of G93A-SOD1 Mice

Previous data indicated a benefit of NDGA when administration is begun at 90 D, but TNFα and its receptor TNF-RI are both upregulated significantly beginning at approximately 80 D of age. Furthermore, 5LOX is increased at 80 D of age. Thus, if 5LOX is involved in the early pathogenesis of ALS, then administration of 5LOX antagonists early in the illness should offer improved benefit. NDGA, which is not currently available for clinical use, is therefore compared with curcumin (a botanical natural product/alternative medical/nutraceutical agent from the curry spice turmeric) and zileuton (zyflo, a clinically available 5LOX antagonist currently used to treat asthma).

Diets and motor performance evaluations. Standard AIN93G rodent diets may be formulated (Dyets, Inc., Bethlehem, Pa.) in one of four ways. Formula one is standard diet with no drug. Formula two contains 0.25% NDGA. Formula 3 contains 0.25% curcumin. Formula four contains 0.1% zileuton. Transgenic mice are trained to the rotarod task at 50 D of age and placed on one of the four diets at 60 D of age. A minimum of 20 animals may be placed on each diet. Rotarod tests may be performed in triplicate trials at weekly intervals, by a technician blinded to the treatment groups. Animal weights are recorded at each test period. Animals are killed when they can no longer perform the rotarod task or right themselves within 10 sec of being placed on their sides. Statistical differences among the drug treatment groups may be assessed using repeated measured analysis of variance (ANOVA) with post-hoc analysis using Student's t-tests at cross-sectional time points. Survivorship curves can be analyzed by logrank methods (Mantel-Haenszel and Kaplan-Meiers statistics). All statistics routines can be performed using GraphPad Prism™ Statistical Applications Software (GraphPad Inc., San Diego, Calif.).

Assays for cytokine transcription, protein oxidation, and leukotrienes. RPA methods, as described above, may be employed in order to assess the effects of the various diets on inflammatory cytokine production. Lumbar-sacral spinal cord tissue can be used for RPA analyses while corresponding cervical-thoracic spinal cords can be used for protein oxidation studies, again according to the methods described above. Each assay compares drug-treated G93A-SOD1 animals at 120 D of age with corresponding G93A-SOD1 animals that had been fed the basal diet without drug supplementation. A minimum of 5 animals may be included in each group for purposes of biochemical analysis.

Leukotrienes A4, B4, C4, D4 and E4 can be assayed in spinal cord lysates using commercially available ELISA systems (Cayman Chemical, San Diego, Calif.). Whole cord lysates are prepared immediately before the assay. In some assays, untreated G93A-SOD1 animals at 80 or 120 D of age are compared to nontransgenic littermates of the same age, in order to assess the effect of G93A-SOD1 transgene expression on leukotriene production. In separate experiments, drug-treated G93A-SOD1 animals are compared to animals receiving a drug-free basal diet, in order to test the efficacy of the several lipoxygenase inhibitors upon leukotriene production within the central nervous system.

Additionally, the same leukotriene ELISA assays can be applied to cortical homogenates and to plasma from the same mice as described above. This allows for the determination of whether each drug enters the central nervous system at a concentration sufficient to inhibit LOX activity in vivo.

It is possible that some leukotrienes may be unstable such that steady-state levels are below the detection limits of the available ELISA assays. In this case, LOX activity can be measured per se by addition of arachidonic acid and 2 mM CaCl₂ to the spinal cord lysate. Resulting formation of LTA₄, LTB₄, and LTC₄ may be measured by ELISA.

Histochemical assessment of gliosis. May be conducted as described previously.

Assessment of SOD1 copy number. The drug treatments can result in decreased expression of the mutant G93A-SOD1 transgene. This may represent a trivial explanation for any protective benefit of the LOX inhibitors. To check for such an artifact, spinal cord lysates from 120D old animals from each drug treatment (including untreated animals) are probed by Western blot methods using monoclonal IgG specific to human SOD1 (Sigma Chemical). Additionally mRNA transcripts for human SOD1 can be monitored by Northern blot methods.

### Example XX

Determination of 5LOX Modulation of TNFα Signaling

The inventors investigated primary microglia and astrocyte cultures from wild-type, G93A-SOD1, and 5LOX knockout mice to determine 5LOX modulation of TNFα signaling; and whether transduction of signals through the TNFα pathway is perturbed by the SOD1 mutation.

Glia of appropriate genotypes are treated with TNFα, and activation is assessed by NO₂⁻ efflux; by phospho-activation of p38 MAP and JNK kinases; and by cytokine profile analysis. This allows for further investigation into the mechanism of action of NDGA by determining whether the compound prevents phosphoactivation of 5LOX in response to TNFα.

Culture of primary microglial cells and astrocytes. Primary mouse microglia can be subcultured from mixed astrocyte-microglial culture by modification of previously described methods (Colton and Gilbert, 1987; Colton et al., 1994; Chan et al., 2001; Cha et al., 2000; Chao et al., 1992). Briefly, the neocortex is removed from 4-6 day old pups under aseptic conditions and large blood vessels removed. Tissue is rinsed and then triturated in cold Ca⁺⁺/Mg⁺ free HBSS buffer. Cells are dispensed into 75 cm² flasks, adjusted to 10⁶/ml in 50% Dulbecco's Modified Essential Medium (DMEM) and 50% F12 media containing 10% heat-inactivated fetal bovine serum, 10% L929 fibroblast-conditioned medium (a source for colony stimulating factor), 1% glutamine, and 1% streptomycin and penicillin. Media is replenished at regular intervals following plating. Microglia are purified from astrocytes by orbital shaking on days 2-4 after initial plating of cells. Astrocytes remain adherent to the plate after orbital shaking, while microglia dissociate into the medium and are replated. This process establishes essentially pure microglial cultures (Colton and Gilbert, 1987; Colton et al., 1994; Chan et al., 2001; Cha et al., 2000; Chao et al., 1992). Purity of cultures may be routinely assessed by immunocytochemistry using fluorescein-conjugated anti-OX-42 antibody to identify microglia, and rhodamine-conjugated anti-glial fibrillary protein (GFAP) antibody to identify astroctyes. Primary glial cultures are prepared from nontransgenic mice, or G93A-SOD1 mice either possessing or lacking functional TNFα and 5LOX genes. Primary astrocytes are prepared from adherent astrocytes, after orbital shaking to remove microglia, as described previously (Robinson et al., 1999).

Stimulation of glial cell cultures. Cells (astrocytes or microglia) are stimulated with recombinant murine TNFα (Calbiochem), at 20 ng/mL and serial ½ dilutions (final concentrations were 20 ng/mL, 10 ng/mL, 5 ng/mL, 2.5 ng/mL, 1.25 ng/mL and 0 ng/mL; subject to modification as necessary). Nitrite is measured in the medium at 24 h, using the standard Griess assay (Physician's Desk Reference, 2002; McGeer and Mcgeer, 2001). ROS production is measured as described below. Additionally, activation of the p38-MAP kinase and JNK pathways is assessed by immunoblot methods using phosphorylation-state specific antibodies to the two MAP kinases (New England Biolabs).

Dose-response curves are compared, for each endpoint, between cellular genotypes (G93A-SOD1 vs. nontransgenic, in both 5LOX^{+/+} vs. 5LOX^{-/-} configurations). Statistical differences in each pair of dose-response curves is determined by 2-factor analysis of variance, with TNFα dosage and cell genotype being considered as independent factors. If 5LOX-ablated cells prove to be deficient in response to TNFα stimulation, experiments may be undertaken to apply exogenous arachidonic acid or individual leukotrienes in order to restore responsivity through bypass of the ablated 5LOX pathway.

Assessment of RNS and ROS generation. Media is removed from cells and NO₂⁻ levels are assayed by the colorimetric Griess diazotization assay (Physician's Desk Reference, 2002; McGeer and McGeer, 2001). Other aliquots of media are analyzed for 3-NO₂-Tyr and 3,3′-diTyr by HPLC with electrochemical array detection using methods optimized previously (Hensley et al., 1998a; Williamson et al., 2002). O₂.⁻ is measured as the ability to reduce nitroblue tetrazolium (NBT) to the blue diformazan, which is colorimetrically detected at 660 nm (Hensley et al., 1998b). For these assays, NBT reduction is measured in the presence and absence of 1000 U/mL bovine erythrocyte SOD1 and the SOD1-inhibitable NBT reduction is used to indicate O₂.⁻. Similar assays may be performed using acetylated cytochrome C (Ac-CytC) as the reducible target, in which case the reduced Ac-CytC is monitored at 555 nm. In separate experiments, ROS generation may be assessed as the change in fluorescence signal from peroxide-dependent oxidation of the fluorogenic substrate dichlorodihydrofluorescein diacetate (H₂DCFDA; Woo, 2000). The DCF analysis may be performed using a 96-well microplate format as well as FACS-based methods. As a final confirmation of radical identity, electron paramagnetic resonance (EPR) spin trapping experiments are performed using 5,5-dimethyl-pyrroline-N-oxide (DMPO) as the spin trap. Authentic superoxide may be generated using a coupled xanthine/xanthine oxidase system. Additionally, adherent microglia are lysed and protein subjected to Western blots using antibodies against 3-NO₂-Tyr. The same lysates are separately derivatized with biotin hydrazide to label protein carbonyls, which is subsequently labeled with streptavidin-conjugated horseradish peroxidase as described above. All blots are developed using enhanced chemiluminescence (ECL).

Assessment of p38-MAPK and JNK pathways. As an additional parameter indicative of TNFα signaling, the phospho-activation of p38-MAPK and JNK pathways may be assessed. Briefly, glial cultures from nontransgenic and G93A-SOD1 mice (in either the 5LOX^{+/+} or 5LOX^{-/-} condition) are stimulated with recombinant murine TNFα. Cells are stimulated in a 24-well format, with TNFα concentrations ranging from 40 ng/mL down to 2.5 ng/mL in serial 1/2 dilutions (4 wells per concentration). Cells are lysed at 5 min, 10 min, 20 min, 30 min, and 60 min after stimulation. The standard lysis buffer contains 10 mM sodium acetate pH 7.4, 0.1 mM orthovanadate and β-glycerophosphate (to inhibit phosphatase and kinase activities) as well as mammalian protease inhibitors (Sigma Chemical). Western blotting is performed using antibodies specific to phosphorylated p38 and JNK, or antibodies directed against non-phosphorylated epitopes (New England Biolabs). Additionally JNK activity is assessed using a pull-down kinase assay (New England Biolabs). Differences in kinetics of stimulation as a function of genotype can be assessed.

When optimum time and dosage parameters are determined for TNFα stimulation of the respective pathways, separate experiments can directly compare G93A-SOD1 and nontransgenic glia (in both 5LOX^{+/+} or 5LOX^{-/-} genotypes). The four genotypes can be stimulated side-by-side, lysed, and blotted on the same membrane in order to determine differences in stimulus sensitivity. This experiment may be replicated and the data statistically assessed using pairwise t-tests. If significant differences are observed between G93A-SOD1 and nontransgenic cells, subsequent experiments can be undertaken to compare wild-type SOD1 expressing glia to G93A-SOD1 expressing glia.

This controls for nonspecific effects of SOD1 over-expression and allows unambiguous determination of whether the SOD1 mutation per se disrupts the TNFα pathway in glial cells.

Cytokine transcription analysis by ribonuclease protection assay. Ribonuclease protection assays (RPAs) have been previously used to monitor redox-sensitive cytokine expression in primary astrocyte and mixed glial cell cultures (Gabbita et al., 2000). This same technique may be used to determine the roles of mutant SOD1 and 5LOX in the modulation of TNFα-stimulated cytokine gene expression. Mixed astrocyte/microglial cultures from nontransgenic and G93A-SOD1 mice (in either the 5LOX^{+/+} or 5LOX^{-/-} condition) are stimulated with recombinant murine TNFα. Cells are lysed at 2, 4, and 6 h after stimulation for purposes of ribonuclease protection assays (RPAs). All four genotypes (G93A-SOD1 and nontransgenic, 5LOX^{+/+} or 5LOX^{-/-}) are compared at each timepoint within the same RPA. A minimum of 4 wells may be used per each treatment, and the RNA pooled. Differences in cytokine levels may be assessed by phosphorimage densitometry with ANOVA and post-hoc t-tests.

Effects of NDGA, zileuton and curcumin on phosphoactivation of 5LOX. As discussed previously, it is possible that NDGA inhibits the phospho-activation of 5LOX as well as the catalytic activity of the enzyme. In order to determine whether this is the case, and whether the 5LOX antagonists zileuton and curcumin act similarly, the following experiments may be performed. EOC-20 cells or primary mixed glial cultures are stimulated with 20 ng/mL TNFα in the presence (or absence) of a minimal IC₁₀₀ concentration of antagonist. After 5-15 min, the cells are lysed and immunoprecipitated with anti-phosphotyrosine or anti-phosphoserine antibodies (Transduction labs). Immunoprecipitates are blotted against anti-5LOX. In the reverse experiment, lysates are immunoprecipitated with anti-5LOX and probed with anti-phosphotyrosine or anti-phosphoserine antibodies.

In using nontransgenic and wild-type human SOD1 expressing cells as a control for G93A-SOD1 expressing cells, it is conceivable that the overexpression of any transgenic protein could alter signal transduction in a manner irrelevant to disease pathology. Fortunately, transgenic mice are available that express wild-type human SOD1 at levels comparable to that of G93A-SOD1 mice. These animals have been previously used as controls, for cytokine expression analyses (Hensley et al., 2002) and no meaningful difference between nontransgenic vs. wildtype human SOD1 expressors was found. In this respect most studies of ALS mice are in excellent agreement: generally nontransgenic and wild type human SOD1-expressing mice are indistinguishable as control animals.

### Example XXI

Correlation of Disease Onset or Progression with Inflammatory Cytokines or Leukotriene Levels in ALS Patients

The inventors determined whether inflammatory cytokines or leukotriene levels are altered in plasma and cerebrospinal fluid of ALS patients, and whether these variables correlated with disease onset or progression. Multiplex antibody arrays are used to simultaneously determine concentrations of 17 cytokines and chemokines (Table IX) while traditional immunoassays are used to assay leukotrienes and prostaglandins. Other BioPlex arrays may also be introduced into the study. This study validates the neuroinflammation hypothesis in human subjects, and identifies specific surrogate biochemical markers of neuroinflammation that can be used to expedite clinical studies of potential ALS therapeutics.

TABLE IX
Protein Level Alterations of Cytokines
in Spinal Cords of G93A-SOD1 Mice.
Data Represent Mean ± SD for 8 Mice
Per Group, Age 120-130 D; *p < 0.05

pg/mg protein

| Analyte | NonTg | G93A-SOD1 | % Change |
|---|---|---|---|
| IL1α | 0.54 ± 0.10 | 1.0 ± 0.10* | 85 |
| IL1β | 120 ± 18 | 164 ± 26* | 37 |
| IL2 | 456 ± 30 | 744 ± 38* | 63 |
| IL3 | 6.2 ± 1.2 | 8.9 ± 1.1* | 44 |
| IL4 | 1.9 ± 0.2 | 2.3 ± 0.1* | 21 |
| IL5 | 438 ± 92 | 595 ± 26* | 36 |
| IL6 | 488 ± 64 | 740 ± 115* | 52 |
| IL10 | 515 ± 71 | 640 ± 35* | 24 |
| IL12p40 | 4.6 ± 0.5 | 5.9 ± 0.8* | 28 |
| IL12p70 | 9.4 ± 1.9 | 13.0 ± 1.6* | 38 |
| IL17 | 2.9 ± 0.22 | 3.1 ± 0.38 | 7 |
| TNFα | 42 ± 7 | 65 ± 3* | 55 |
| IFNγ | 1063 ± 96 | 1500 ± 91* | 41 |
| KC | 5.8 ± 1.1 | 8.9 ± 1.5* | 53 |
| MIP-1α | 248 ± 43 | 323 ± 41* | 30 |
| RANTES | 17 ± 3 | 34 ± 6* | 100 |
| GM-CSF | 1055 ± 54 | 1113 ± 57 | 5 |

Evaluation of human ALS-afflicted subjects may be used to further support the data if a 5LOX-TNFα axis is implicated as a meaningful component of ALS pathogenesis in the SOD1 mutant mouse. The most straightforward means of doing so would be to measure leukotriene levels, 5LOX, and TNFα in human CNS and peripheral tissue. In point of fact, both TNFα and soluble TNF-RI have been found modestly elevated in serum from ALS-afflicted humans (Poloni et al., 2000). Leukotriene levels have not been well-measured or correlated with clinical parameters; nor have most other cytokines and chemokines.

Clinical ALS samples and clinical parameters. Plasma and cerebrospinal fluid (CSF) from ALS-afflicted persons can be collected and archived. Samples collected may be stored at −80° C. until analyzed. The patients with ALS must have a clinical diagnosis of probable, definite, sporadic or familial ALS and be older than 18 years of age. The control subjects must have a lumbar puncture for standard, clinical indications. No control subject undergoes a spinal tap specifically for this study. All subjects must be willing and able to give informed consent. Based on past rates of patient acquisition and current census, it is estimated that 200 or more ALS patients can be analyzed over a five-year period. Patients will be assessed routinely (at 2 month intervals) for respiratory capacity (forced vital lung capacity, FVC) and standard-of-living indices (ALSFRS, discussed below). All patients will be followed for survival.

Protocols/instruments for clinical evaluation. A comprehensive evaluation for each patient at baseline and each follow-up visit may be completed. Age of onset, date of onset, site of onset, gender, family history, medications including experimental agents and vitamins, and past medical history will be obtained. To assess rate of disease progression, measures of pulmonary function (forced vital capacity, FVC) and the ALS functional rating scale (ALSFRS) score may be obtained at each visit. Both measures are well correlated with disease severity and survival (Andres et al., 1987; Andres et al., 1988). The ALS functional rating scale (ALSFRS) is a widely accepted functional rating test. It is a quickly administered (five minute) ordinal rating scale (ratings 0-4) used to determine patients' assessment of their capability and independence in 10 functional activities. All 10 activities are relevant in ALS. Initial validity was established by documenting that change in ALSFRS scores in ALS patients correlated with change in strength over time, as measured by the Tufts Quantitative Neuromuscular Examination (Cedarbaum, 1996; Cedarbaum et al., 1994).

Control samples: “Normal”, “non-neurological disease” and “neurological disease” comparator groups. ALS plasma can be compared to three types of control tissue, taken from (1) “normal” nondiseased individuals age-matched to ALS subjects; (2) acutely hospitalized (i.e., ill) patients who do not suffer from a neurological disorder, or from conditions likely to cause pronounced systemic inflammation; (3) patients with Alzheimer's disease or other neurological disorders distinct from ALS who are not suffering from severe, acute peripheral pathologies. Additionally efforts may be made to collect tissue from patients suffering from frank inflammatory conditions such as sepsis or severe rheumatic disorders; these represent a type of positive control population. Up to 200 ALS and 200 control subjects may be sampled for analysis of plasma. Additionally, up to 150 ALS and 150 neurological diseased (non-ALS) subjects may be sampled for cerebrospinal fluid.

ELISA assays. Competitive enzyme linked immunosorbent assays (ELISAs) are commercially available for all the major leukotrienes (LTB₄, LTC₄, LTD₄, LTE₄ all manufactured by Cayman Chemical, San Diego, Calif.) and the major prostaglandin for comparison, PGE₂ (also supplied by Cayman Chemical). Such ELISAs have been evaluated for eicosanoid mediators in normal plasma and plasma from septic humans and animals. The sensitivity of the ELISAs is sufficient to measure baseline levels of cysteinyl leukotrienes in normal human plasma, and clear elevations are observed during periods of sepsis (Quinn et al., 1996). Additionally, ELISAs may be employed for quantitation of TNFα and C-reactive protein (CRP, a major acute-phase reactant that is almost universally elevated in classical inflammatory conditions).

Cytokine profile analysis using BioPlex arrays. The BioPlex technology is described herein. This microbead-based antibody array system allows for the simultaneous quantitation of 17 cytokines and chemokines simultaneously, using as little as 0.2 mL of sample. BioPlex analysis may be performed on ALS and comparator populations, for cytokine/chemokine profiles in plasma and CSF. Human-specific antibody sets may be used, representing pro- and anti-inflammatory cytokines and chemokines identical or analogous to the murine species listed in Table IX. Specific analytes to be assayed are: TNFα, TNF-RI, IL1β, IL2, IL4, IL5, IL6, IL7, IL10, IL12p70, IL13, IL17, GCSF, GM-CSF, IFNγ, MCP1, and MIP1β. When combined with data from classical ELISA assays, this effort represents the most thorough survey to date of inflammatory biomarkers in a well-documented group of individuals suffering from defined neurological disease.

Statistical analysis: Data preprocessing and quality control. Data obtained from each assay is analyzed for statistical differences between ALS and comparator populations. It is possible that this comparison may be hindered by analyte decomposition as a function of storage age. To assess this possibility several tactics may be employed. First, leukotriene standards may be spiked into fresh normal plasma (or CSF) and aliquots stored frozen at −80° C. for various time periods (0 D, 1 week, 2 weeks, 1 month, 2 months, 4 months, 8 months, 1 year, 2 years). These spiked samples may be assayed at regular intervals to determine storage stability. If analyte decomposition is noted, the deterioration rate may be evaluated in general first-and second-order kinetic models. Data from clinical samples may then be corrected based on knowledge of the collection date and storage period, after which statistical differences between groups may be assessed.

In a second strategy to correct for storage-time artifacts, the raw ELISA data from each group (normal and ALS) may be regressed against storage time for the individual samples. General least-squares curve fitting may be employed to determine the time dependence, if any, of sample deterioration for the several analytes. Again the data may be transformed accordingly to correct for any significant storage-time correlations. As a final quality assurance measure, a single reference sample may be analyzed repeatedly with each group of clinical samples. This allows the determination of inter-assay variability.

Statistical analysis of group-specific differences. Disease-specific differences amongst the several groups (ALS, “normal” control, neurological disease controls, and non-neurological disease controls) may be assessed by generalized analysis of variance (ANOVA) procedures followed by appropriate post-hoc analyses (principally Bonferonni methods and Mann-Whitney tests). Statistical analyses may be performed using GraphPad Prism™ statistical analysis software (GraphPad Inc., San Diego, Calif.) and other commercially available programs, as necessary.

Correlation of biochemical measures with clinical parameters. Because samples are obtained from ALS patients throughout the course of disease, it is possible to explore relationships among the biochemical variables and clinical parameters such as FVC or duration of illness at the time of sample collection. These correlations can be assessed for statistical significance using Spearman's rank tests (nonparametric) and Pearson correlation analysis (parametric). This allows for the identification of biochemical variables that best predict clinical status. Care is taken to note any variation of analyte levels associated with NSAID or other drug use by the ALS patients.

Furthermore, plasma and CSF data obtained from the same patients can be assessed for statistical correlation to determine whether peripheral levels of the leukotrienes can predict levels in the CNS. This allows for the identification of surrogate markers for neurological damage that can be utilized as intermediate endpoints in any future clinical studies of ALS therapeutics. All correlation and regression analyses can be performed using GraphPad Prism™ Statistical Analysis software (GraphPad Inc., San Diego, Calif.).

Chemometric analysis and creation of disease-discriminant functions. This study allows for very efficient collection of data on a large number of distinct analytes (at least 21 inflammatory proteins, cytokines, chemokines, eicosanoids and prostaglandins). It is possible that neurological disease in ALS patients can not be clearly indexed by any one single variable. Nonetheless, parameters indicative of disease severity might be extracted from the overall data matrix by considering subtle relationships amongst several individual analytes simultaneously. The scope of this study presents a unique opportunity for advanced chemometric analysis of inflammatory reactions in the context of neurodisease, with the goal of defining disease-discriminant functions constructed from linear combinations of independent analyte variables.

The main tool employed in the chemometric exploration of human biochemical data is principal component analysis (PCA; as described by (Otto, 1999). PCA is a mathematical technique for decomposing a large data matrix into a product of two smaller matrices, a scoring matrix and a loading matrix. Manipulation of the component matrices yields a series of “principal components” (PCs) which are linear combinations of the original variables. Hence the principal components are reconstructed variables that have two key properties. First, PCs are uncorrelated (or independent) of one another. Biologically this means that the PCs are not likely to represent mutually dependent entities that simply autocorrelate. Second, the PCs are reconstructed to maximize variance. The first principal component implicitly contains most of the variance in the original data set; the second PC contains less, and so forth. This is important because analytes that display little variation amongst individuals are likely to be poor prognostic indicators.

Each data point (originally containing any large number of analyte measurements) is thus transformed into a single coordinate containing one, two, or three PCs. Because a large (often a majority) fraction of variance in a data set can be described by one, two, or three PCs, a plot, of each transformed point in 2- or 3-dimensional space allows convenient visualization of the original complex data set. In a successful PCA, the various subpopulations contributing to the original data matrix (for example, normal vs. ALS groups) become spatially resolved in the PC transform.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

References

The following references, to the extent that they provide exemplary procedural or other details supplementary to those set forth herein, are specifically incorporated herein by reference.

U.S. Pat. No. 4,708,964

U.S. Pat. No. 4,857,558

U.S. Pat. No. 5,047,593

U.S. Pat. No. 5,068,251

U.S. Pat. No. 5,208,262

U.S. Pat. No. 5,569,649

U.S. Pat. No. 5,891,924

U.S. Pat. No. 6,235,287

U.S. Pat. No. 6,264,995

Akimoto et al., Ann. Nutr. Metab., 37:218-224, 1993.

Alexianu et al., Neurol., 57:1282-1289, 2001.

Andres et al., Neurol., 38:405-408, 1988.

Andres et al., Neurologic Clinics, 5:125-141, 1987.

Archer, FASEB J., 7:349-360, 1993.

Balestrero et al., Int. J. Dermatol., 40:474-475, 2001.

Barany and Merrifield, In: The Peptides, Gross and Meienhofer (Eds.), Academic Press, NY, 1-284 1979.

Beal et al., J. Neurosci., 13:4181-4192, 1993.

Bensimon et al., J. Neurol., 249:609-615, 2002.

Birt et al., Pharmacol. Therapeut., 90:157-177, 2001.

Blum et al., Biochemistry, 39:15705-15712, 2000.

Cedarbaum et al., Neurol., 44(2):A256, 1994.

Cedarbaum, Archives of Neurology, 53(2):141-147, 1996.

Cha et al., Brain Res., 853:156-161, 2000.

Chan et al., Glia, 22:87-95, 2001.

Chao et al., J. Immunol., 149:2736-2641, 1992.

Chen et al., Nature Med., 6:797-801, 2000.

Chen et al., Nature Med., 6:797-801, 2000.

Chen et al., Nature, 372:179-182, 1994.

Chi et al., Biochem. Pharmacol,. 62:1185-1192, 2001.

Cho et al., Tox. App. Pharmacol., 168:64-71, 2000.

Chu et al., J. Neurosci., 22:1763-1771, 2002.

Colton and Gilbert, FEBS Lett., 223:284-288, 1987.

Colton et al., Ann. NY Acad. Sci,. 738L:54-63; 1994.

Cranston et al., Fed. Proc., 6:318, 1947.

Damnis, Geriatrics,57:46-50, 2002.

Deng et al., Science, 20:1047-1051, 1993.

Drachman et al, Ann. Neurol., 52:771-772, 2002.

Floyd et al., J. Neural. Transmission Suppl., 60:387-414, 2000.

Frautschy et al., Neurobiol. Aging, 22:993-1005, 2001.

Fukuda et al., Agric. Biol. Chem., 49:301-306, 1985.

Funk, Science, 294:1871-1875, 2001.

Gabbita et al., Arch. Biochem. Biophys., 376:1-13, 2000.

Gazit et al., J. Med. Chem., 32:2344-2352, 1989.

Gupta et al., Pharmacol. Biochem. Behav., 71:245-249, 2002.

Gurney et al., Annals Neurol., 39:147-157, 1996.

Hall et al., Glia, 23: 249-256, 1998.

Hellenbeck, Nature Med., 8:1363-1368, 2002.

Hensley et al., J. Neurochem., 65:2146-2156, 1995.

Hensley et al., J. Neurochem., 71:2549-2557, 1998a.

Hensley et al., J. Neurosci., 18:8126-8132, 1998b.

Hensley et al., J. Neurochem., 82:365-374, 2002.

Hirose et al., J. Lipid Res., 32:629-638, 1991.

Hirose et al., Anticancer Res., 12:1259-1266, 1992.

Horoszewicz et al., Cancer Res., 43:1809-1818, 1983.

Hostettmann and Marston, Prog. Clin. Biol. Res., 213:43-51, 1986.

Huang et al., Life Sci., 69:1057-1065, 2001.

Hwu et al., J. Med. Chem., 41:2994-3000, 1998.

Johnson et al., Proc. Natl. Acad. Sci. USA, 97:12571-12576, 2000.

Kageura et al., Bioorganic Med. Chem., 9:1887-1893; 2001.

Kaul et al., Nature, 410:988-994, 2001.

Kita et al., Biol. Pharm. Bull., 18:1283-1285, 1995.

Knopman, Curr. Neurol. Neurosci. Rep., 1:428-434, 2001.

Lehman et al., Advanc. Food Res., 3:197, 1951.

Lim et al., J. Neurosci., 21:8370-8377, 2001.

Little and O'Callagha, Neurotox., 22:607-618, 2001.

Madamanchi et al., Oncogene, 16:417-422, 1998.

Marzinzig et al., Nitric Oxide: Biology and Chemistry, 1:177-189, 1997.

Massiot et al., J. Chem. Soc. Perkin Trans., 1:3071-3079, 1988.

Matsumura et al., Biol. Pharm. Bull., 18:1016-1019, 1995.

Matsumura et al., Biol. Pharm. Bull., 21:469-473, 1998.

Mcgeer and McGeer, Neurobiol. Aging, 22:799-809, 2001.

McMurray, TINS, 24(suppl):S32-S38, 2001.

Meda et al., Nature, 374:647-650, 1995.

Merrifield, Science, 232: 341-347, 1986.

Miller, Amyotroph. Lateral. Scler. Other Motor Neuron Disord., 2:3-7, 2001.

Miquel et al., Archives Gerontol. Geriat., 34:37-46, 2002.

Mirzoeva and Calder, Prost. Leuko. Essen. Fatty Acids, 55:441-449, 1996.

Morgan et al., Nature, 408:982-985, 2000.

Nakana et al., Biol. Pharm. Bull., 25(9):1247-1249, 2002.

Osawa et al., Agric. Biol. Chem., 49:3351-3352, 1985.

Otto, In: Chemomoetrics, Wiley-VCH, Veinheim, Germany, 1999.

Park et al., FEBS Lett., 465:93-97, 2000.

Pasparakis et al., J. Exp. Med., 184:1397-1411, 1996.

Physician's Desk Reference, 56^{th} Ed., Medical Economics Company, Inc., Nontvale, N.H., 772-776; 1862-1866, 2002.

Poloni et al., Neurosci. Lett., 287:211-214, 2000.

Quinn et al., Shock, 6:142-149, 1996.

Rajakrishnan et al, Phytother. Res., 13:571-574, 1999.

Remington's Pharmaceutical Sciences, 18th Ed. Mack Printing Company, 1990

Rizzo and Carlo-Stella, Blood, 88:3792-37800; 1996.

Robinson et al., J. Neurosci. Res., 55:724-732, 1999.

Rosen et al., Nature, 362:59-62, 1993.

Shimizu et al., Lipids, 26:512-516, 1991.

Shishido et al., Pharmacol., Biochem. Behav., 69:469-474, 2001.

Soliman and Mazzio, Proc. Soc. Exp. Biol. Med., 218:390-397, 1998.

Stewart and Young, In: Solid Phase Peptide Synthesis, 2d. ed., Pierce Chemical Co., 1984.

Stewart et al., Arch. Biochem. Biophys., 365:71-74, 1999.

Talvani et al., Infec. Immunol., 70:4247-4253, 2002.

Tam et al., J. Am. Chem. Soc., 105:6442, 1983.

Thakkar et al., J. Med. Chem., 36: 2950-2955, 1993.

Thalmann et al., Prostate, 44: 91-104, 2000.

Tikka et al., J. Neurosci., 21:2580-2588, 2001.

Vila et al., Curr. Opin. Neurol., 14:483-489, 2001.

Virgili and Contestabile, Neurosci. Lett., 10:123-126, 2000.

Walker et al., J. Neuroimmunol., 63:163-174; 1995.

Wieder et al., Leukemia, 15:1735-1742, 2001.

Williamson et al., Nitric Oxide: Biol. Chem., 6:221-227, 2002.

Wisniewski et al., Am. J. Med. Genet. Supp., 7:287-297, 1990.

Wolter et al., J. Nutr., 132:298-302, 2002.

Woo et al., J. Biol. Chem., 275:32357-32362, 2000.

Wu et al., J. Neurosci., 22:1763-1771, 2002.

Yang et al., Biochem. Biophys. Res. Commun., 245(2):435-438, 1998.

Yoshihara et al., J. Neurochemistry, 80,158-167, 2002.

Yijanheikki et al., Proc. Natl. Acad. Sci. USA, 96:13496-13500, 1999.

Zhu et al., Nature, 417:74-78, 2002.

What is claimed:

1. A method of inhibiting an inflammatory disease in a subject comprising providing to said subject an effective amount of tethered bis(polyhydroxyphenyl) compounds or O-alkyl derivatives thereof.

2. The method of claim 1, wherein the tethered bis(polyhydroxyphenyl) compounds have the general formula: 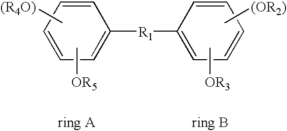
wherein R₁ is an alkyl chain of at least 2 and less that 10 carbon units in length, and wherein R₂-R₅ are —H atoms or alkyl chains comprising one or more carbon atoms.

3. The method of claim 2, wherein R₁ comprises a structural motifs selected from C═C bonds; alkynes; amide ester, ether or sulfide linkages; intervening ring structures; ketone moieties; or halogenated side chain.

4. The method of claim 2, wherein the alkyl chains of R₂-R₅ further comprise of the group selected from halogens, carbonyl groups, boronate esters and closed ring structures.

5. The method of claim 2, wherein at least one of OR₄ and OR₅ and at least one of OR₂ and OR₃ is a hydroxyl or alkoxyl group.

6. The method of claim 2, wherein the tethered R₁ is a branched chain hydrocarbon.

7. The method of claim 5, wherein at least three of OR₂-OR₅ is a hydroxyl or alkoxyl group.

8. The method of claim 1, wherein the disease is a neurological disease, a cancer or hyperplasia.

9. The method of claim 1, wherein the neurological diseases comprises pro-inflammatory cytokine stimulation of a cell.

10. The method of claim 1, wherein the tethered bis(polyhydroxyphenyl) compound is nordihydroguaiaretic acid (NDGA) or O-alkyl derivatives thereof or pro-drugs of the same.

11. The method of claim 1, wherein the tethered bis(polyhydroxyphenyl) compound is piceatannol or O-alkyl derivatives thereof or pro-drugs of the same.

12. The method of claim 1, wherein the tethered bis(polyhydroxyphenyl) compound is resveratrol or O-alkyl derivatives thereof or pro-drugs of the same.

13. The method of claim 1, wherein the tethered bis(polyhydroxyphenyl) compound is curcumin or O-alkyl derivatives thereof or pro-drugs of the same.

14. The method of claim 1, wherein the tethered bis(polyhydroxyphenyl) compound is a reduced curcumin or O-alkyl derivatives thereof or pro-drugs of the same.

15. The method of claim 14, wherein the reduced curcumin is dihydrocurcumin or tetrahydrocurcumin, or O-alkyl derivatives thereof or pro-drugs of the same.

16. The method of claim 1, wherein the tethered bis(polyhydroxyphenyl) compound is rooperol or O-alkyl derivatives thereof or pro-drugs of the same.

17. The method of claim 1, wherein the tethered bis(polyhydroxyphenyl) compound is rosmarinic acid or O-alkyl derivatives thereof or pro-drugs of the same.

18. The method of claim 1, wherein the tethered bis(polyhydroxyphenyl) compound is a tyrphostin comprising two phenolic ring structures, or O-alkyl derivatives thereof or pro-drugs of the same.

19. The method of claim 1, wherein the tethered bis(polyhydroxyphenyl) compound is butein or O-alkyl derivatives thereof or pro-drugs of the same.

20. The method of claim 1, wherein the tethered bis(polyhydroxyphenyl) compound is sesamin or O-alkyl derivatives thereof or pro-drugs of the same.

21. The method of claim 1, wherein the tethered bis(polyhydroxyphenyl) compound is a sesame composition or O-alkyl derivatives thereof or pro-drugs of the same.

22. The method of claim 21, wherein the sesame composition is sesame oil or sesame seed extract, or O-alkyl derivatives thereof or pro-drugs of the same.

23. The method of claim 8, wherein the neurological disease is amyotrophic lateral sclerosis (ALS) (familial or sporadic).

24. The method of claim 8, wherein the neurological disease is motor neuron disease (NND) of similar clinical presentation to ALS.

25. The method of claim 8, wherein the neurological disease is Alzheimer's disease (AD).

26. The method of claim 8, wherein the neurological disease is Parkinson's disease (PD).

27. The method of claim 8, wherein the neurological disease is multiple sclerosis (MS).

28. The method of claim 8, wherein the neurological disease is myasthenia gravis (MG).

29. The method of claim 8, wherein the neurological disease is Huntington's disease (HD).

30. The method of claim 8, wherein the neurological disease is spinal-bulbar atrophy (SBA).

31. The method of claim 8, wherein the neurological disease is frontal-temporal dementia (FTD).

32. The method of claim 8, wherein the neurological disease is stroke (ischemia-reperfusion injury of the brain).

33. The method of claim 8, wherein the neurological disease is encephalomyelitis or meningitis.

34. The method of claim 8, wherein the neurological disease is traumatic brain injury.

35. The method of claim 8, wherein the neurological disease is retinal degeneration.

36. The method of claim 8, wherein the neurological disease is HIV-associated dementia.

37. The method of claim 9, wherein the cell is a microglial cell.

38. The method of claim 9, wherein the cell is a neuron.

39. The method of claim 9, wherein the cell is a macrophage type cell, a Kupffer cell, Mueller cell or other myeloid cell.

40. A method of treating inflammatory diseases or cancers or hyperplasias in a subject comprising providing to said subject an effective amount of a bis(polyhydroxyphenyl) compound or O-alkyl derivatives thereof to inhibit pro-inflammatory cytokine action on macrophage-like cells.

41. The method of claim 40, wherein the inflammatory disease is cancer or hyperplasia of the eyes, respiratory system, musculo-skeletal system, lymphatic system, reticulo-endothelial system, hepatic system, prostrate, breast, colon, reproductive, urinary or alimentary tract.

42. The method of claim 40, wherein the inflammatory disease is chronic inflammatory or rheumatic diseases.

43. The method of claim 40, wherein said inflammatory or rheumatic disease is arthritis, inflammatory or rheumatic diseases of the eye, or diseases of the respiratory or musculo-skeletal system, or alimentary tract.

44. A method of treating a subject with neurological diseases, cancers or hyperplasias comprising administering to said subject an effective amount of a bis(polyhydroxyphenyl) or O-alkyl derivatives thereof to inhibit microglial activation.

45. The method of claim 44, wherein administration is orally, subcutaneously, intrathecally, by inhalation, injection, microprojectile bombardment, intravenously, or topically.

46. A method for enhancing the efficacy of non-bis(polyhydroxyphenyl) neuropharmaceuticals comprising providing to a subject said non-bis(polyhydroxyphenyl) neuropharmaceutical and a bis(polyhydroxyphenyl) or O-alkyl derivative thereof.

47. The method of claim 46, wherein the non-bis(polyhydroxyphenyl) neuropharmaceutical is riluzole.

48. The method of claim 46, wherein the non-bis(polyhydroxyphenyl) neuropharmaceutical is minocycline.

49. The method of claim 46, wherein said non-bis(polyhydroxyphenyl) or bis(polyhydroxyphenyl) or O-alkyl derivative thereof is provided gradually over a time period of greater than one minute.

50. The method of claim 46, wherein said non-bis(polyhydroxyphenyl) or bis(polyhydroxyphenyl) or O-alkyl derivative thereof is provided gradually over a time period of greater than ten minutes.

51. The method of claim 46, wherein said non-bis(polyhydroxyphenyl) or bis(polyhydroxyphenyl) or O-alkyl derivative thereof is provided gradually over a time period of greater than thirty minutes.

52. The method of claim 46, wherein said non-bis(polyhydroxyphenyl) or bis(polyhydroxyphenyl) or O-alkyl derivative thereof is provided gradually over a time period of greater than sixty minutes.

53. The method of claim 46, wherein said non-bis(polyhydroxyphenyl) or bis(polyhydroxyphenyl) or O-alkyl derivative thereof is provided gradually over a time period of greater than one-hundred twenty minutes.

54. The method of claim 46, wherein said non-bis(polyhydroxyphenyl) or bis(polyhydroxyphenyl) or O-alkyl derivative thereof is provided gradually over a time period of greater than four hours.

55. The method of claim 46, wherein said non-bis(polyhydroxyphenyl) or bis(polyhydroxyphenyl) or O-alkyl derivative thereof is provided gradually over a time period of greater than eight hours.

56. The method of claim 46, wherein said non-bis(polyhydroxyphenyl) or bis(polyhydroxyphenyl) or O-alkyl derivative thereof is provided gradually over a time period of greater than twelve eight hours.

57. The method of claim 46, wherein said non-bis(polyhydroxyphenyl) or bis(polyhydroxyphenyl) or O-alkyl derivative thereof is provided gradually over a time period of greater than twenty-four hours.

58. The method of claim 46, wherein said bis(polyhydroxyphenyl) or O-alkyl derivative thereof is provided more than once.

59. The method of claim 46, wherein said non-bis(polyhydroxyphenyl) is provided more than once.

60. The method of claim 46, wherein said bis(polyhydroxyphenyl) or O-alkyl derivative thereof is provided before the non-bis(polyhydroxyphenyl).

61. The method of claim 46, wherein said bis(polyhydroxyphenyl) or O-alkyl derivative thereof is provided at the same time as the non-bis(polyhydroxyphenyl).

62. The method of claim 46, wherein said bis(polyhydroxyphenyl) or O-alkyl derivative thereof is provided after the non-bis(polyhydroxyphenyl).
